(19) United States
(12) Patent Application Publication
(10) Pub. No.: US 2026/0250322 A1
(43) Pub. Date: Aug. 27, 2026

(54) NEW PEPTIDES AS SELECTIVE IL-23 RECEPTOR INHIBITORS

(71) Applicant: SANOFI, Paris (FR)

(72) Inventors: Thomas BOEHME, Frankfurt Am Main (DE); Melissa BESENIUS, Frankfurt Am Main (DE); Andreas EVERS, Frankfurt Am Main (DE); Torsten HAACK, Frankfurt Am Main (DE); Christoph POEVERLEIN, Frankfurt Am Main (DE); Garima TIWARI, Frankfurt Am Main (DE); Michael WAGNER, Frankfurt Am Main (DE)

(21) Appl. No.: 19/558,815

(22) Filed: Mar. 6, 2026

Related U.S. Application Data
(63) Continuation of application No. PCT/EP2024/074933, filed on Sep. 6, 2024.

(30) Foreign Application Priority Data
Sep. 8, 2023 (EP) 23306502.8

(51) Int. Cl. C07K 7/64; A61K 38/00

(52) U.S. Cl. CPC C07K 7/64; A61K 38/00

(57) ABSTRACT

The disclosure relates to IL-23 receptor inhibitor compounds of formula (I), their pharmaceutical compositions, and their medical uses, for example in the treatment of inflammatory bowel diseases such as Crohn's disease.

### REFERENCE TO SEQUENCE LISTING

The sequence listing submitted Mar. 6, 2026, as an xml file named 11951-003US1_ST26.xml, created on Mar. 4, 2026, and having a size of 166,235 bytes is herein incorporated by reference pursuant to 37 C.F.R. § 1.52 (e) (5).

### FIELD

The disclosure relates to peptide IL-23 receptor inhibitors and their medical use, for example in the treatment of inflammatory bowel diseases such as Crohn's disease. More specifically, relates to macrolactams useful as IL-23 receptor inhibitors.

### BACKGROUND

There is increasing evidence that interleukin-23 (IL-23) cytokine is playing a crucial role in the pathogenesis of autoimmune inflammation and related diseases and disorders, such as asthma, rheumatoid arthritis, psoriasis, multiple sclerosis and inflammatory bowel diseases (IBDs), e.g., ulcerative colitis and Crohn's disease. The primary role of IL-23R could be revealed by acute and chronic mouse models of IBD and downstream effector cytokines in disease pathogenesis. Th17 cells, y8 T cells, natural killer (NK) cells, dendritic cells, macrophages, and innate lymphoid cells, which are found abundantly in the intestine are adaptive and innate immune cells which express IL-23R. IBD patients have an increased gene expression and IL-23R level at the intestine mucosal surface. There is evidence that IL-23 contributes to this effect by promoting the development of a pathogenic CD4+ T cell population that produces IL-6, IL-17, and tumor necrosis factor (TNF).

Concentration of IL-23 is increased in the intestine, where it is thought to play a key role in regulating the balance between tolerance and immunity through T-cell-dependent and T-cell-independent pathways of intestinal inflammation. This regulation has effects on T-helper 1 (Th1) and Th17-associated cytokines, as well as repressing regulatory T-cell responses in the gut, promoting inflammation. Furthermore, IL-23R polymorphisms have been associated with susceptibility to gut inflammation, further confirming the importance of the IL-23 pathway in intestinal homeostasis.

Psoriasis is a chronic skin disease with a prevalence in the Caucasian population of about 2%-3% has been shown to be mediated by the body's T cell inflammatory response mechanisms. Via the induction of interleukin-17, activation of macrophages and regulation of T memory cells, IL-23 is one of several interleukins implicated as a key player in the pathogenesis of psoriasis by maintaining chronic autoimmune inflammation. Neutralizing antibodies against IL-23 showed IL-23-dependent inhibition of psoriasis development in animal models of psoriasis while high expression of IL-23 and IL-23R has been demonstrated to be increased in tissues of patients with psoriasis.

IL-23 is a part of the IL-12 family of cytokines and is composed of a p19 subunit specific to IL-23 and the p40 subunit of IL-12, which is a cytokine involved in the development of interferon-γ (IFN-γ)-producing T helper 1 (TH1) cells. Although IL-23 and IL-12 both contain the p40 subunit, they have different phenotypic properties. While animals with deficiency in IL-12 are susceptible to inflammatory autoimmune diseases, IL-23 deficient animals are resistant, most likely due to a reduced number of CD4+ T cells producing IL-6, IL-17, and TNF in the CNS. IL-23 binds to IL-23R, which is a heterodimeric receptor composed of IL-12Rβ1 and IL-23R subunits. Binding of IL-23 to IL-23R activates the Jak-Stat signaling molecules, Jak2, Tyk2, Stat1, Stat3, Stat4, and Stat5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

Based on the biological importance, inhibition of the IL-23 pathway is an attractive option for treatment of IL-23-related diseases and disorders. A number of antibodies that bind to IL-23 or IL-23R have been identified, including ustekinumab, a humanized antibody that binds IL-23, which has been approved for the treatment of psoriasis. More recently, polypeptide inhibitors that bind to IL-23R and inhibit the binding of IL-23 to IL-23R have been identified (see, e.g., US Patent Application Publication No. US2013/0029907). Clinical trials in Crohn's Disease or psoriasis with ustekinumab and briakinumab (which target the common p40 subunit) and tildrakizumab, guselkumab, brazikumab, and risankizumab (which target the unique p19 subunit of IL-23) highlight the potential of IL-23 signaling blockade in treatment of human inflammatory diseases.

While these findings are promising, challenges remain with respect to identifying stable and selective agents that preferentially target the IL-23 pathway in the intestine, which can be used for the treatment of intestinal inflammation, such as intestinal bowel diseases, including Crohn's disease, ulcerative colitis and related disorders.

Irritable bowel disease (IBD) patients with autoimmune inflammation in the intestinal tract can benefit from an oral-to-local treatment where compounds specifically target the IL-23 pathway from the luminal side of the gut.

A problem associated with the use of compounds as an orally administered therapeutic in the treatment of inflammatory diseases and other indications can be their limited stability in the intestinal environment.

Therefore, in developing new therapeutic molecules, there is a need for variants with improved pharmaceutical properties, for example increased stability against proteases present in the gut and/or increased chemical or physical stability and/or a prolonged half-life in vivo and/or increased potency/efficacy in vivo.

Also, there remains a need for anti-inflammatory therapies that avoid or alleviate the common systemic side effects of IL-23R based therapies (e.g., defense against infections), thereby achieving an anti-inflammatory effect with improved tolerability.

US 2013-029907 A1 discloses linear peptide inhibitors of the IL-23 receptor.

Protagonist's WO 2016/011208 A1, WO 2017/011820 A2, WO 2018/089693 A2, WO 2018/022937 A1, WO 2018/136646 A1 and WO 2022/109328 A1, as well as Sayago et al. (ACS Med. Chem. Lett. 2018, 9, 912-916) disclose peptide inhibitors of the IL-23 receptor for oral administration. A common structural characteristic of the disclosed compounds is a ring formed by 6 amino acids as ring members.

WO 2013/063468 discloses cyclic peptides which are modified by long chain hydrocarbon groups resulting in amphiphilic molecules useful as drug delivery system including nucleotide delivery to cells.

WO 2015/179438 A1 also discloses cyclic peptides of a 2 to 10 membered ring formed by amino acids as inhibitors Rac or Rho in a cell or tissue, wherein at least two amino acids are arginine.

Quiniou et al. (Am J Physiol Regul Integr Comp Physiol, 2014, 307: R1216-R1230) disclose small peptide non-competitive inhibitors made of D-amino acids.

Kuchar et al. (Proteins, 2013) disclose IL-23 receptor inhibitors derived from three-helix bundle scaffold of the albumin-binding domain of streptococcal protein G.

WO 2023/288028 discloses cyclic peptide inhibitors of IL-23 receptor of 16 amino acid residues with a cyclization obtained with a disulfide bond between residues at positions 4 and 9.

WO 2023/288019 discloses cyclic peptide inhibitors of IL-23 receptor of 16 amino acid residues with a cyclization obtained with a disulfide or thioether bond between residues at positions 4 and 9.

WO 2023/288017 discloses bicyclic peptide inhibitor of IL-23 receptor with bicyclization obtained with a first disulfide or thioether bond between a first pair of amino acid residues and a second amide or thioether bond between a second pair of amino acid residue.

Therefore, there is still a need for improved IL-23 receptor inhibitor compounds for treating and/or preventing autoimmune or inflammatory disease.

There is a need for IL-23 receptor inhibitor compounds with improved in vitro and in vivo stability.

There is a need for IL-23 receptor inhibitor compounds with improved oral bioavailability and/or stability upon oral administration.

There is a need for IL-23 receptor inhibitor compounds which selectively bind to the IL-23 receptor in the intestine.

There is a need for compounds with improved IL-23 receptor inhibitor properties.

There is a need for IL-23 receptor inhibitor compounds with high binding affinity.

There is a need for compounds able to inhibit the IL-23 receptor with an IC₅₀ in the nanomolar range, for example equal or below 50 nM, or again equal or below 20 nM.

There is a need for IL-23 receptor inhibitor compounds and with improved protease stability.

The disclosure provides peptide compounds of the formula (I) that bind to IL-23 receptor (IL-23R), and in some embodiments are suitable for oral administration, and/or address one or more of the above-referenced needs.

Therefore, the disclosure provides compounds of formula (I) having high affinity to the IL-23 receptor.

### SUMMARY

The present disclosure relates to a peptide compound of formula (I):

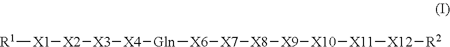

wherein
R¹ is —H or acetyl;
X1 is an amino acid residue selected among a residue of Asp, D-Asp, Glu, D-Glu, Lys, D-Lys, Orn, D-Orn, Ahx and Apt;
X2 is an amino acid residue selected among a residue of Asn, D-Asn, Arg and Gln;
X3 is an amino acid residue selected among a residue of His, Lys, Arg, Asn, Gln, Trp and Thr;
X4 is an amino acid residue selected among a residue of Trp, Wme, Nak, Wcl and Wfl;
X6 is an amino acid residue selected among a residue of Arg, Leu, Ala, Val, Glu, Aib and Agb;
X7 is an amino acid residue selected among a residue of Yme, Tyr, Trp and Yae;
X8 is an amino acid residue selected among a residue of Nal, Nak and Trp;
X9 is an amino acid residue selected among a residue of Arg, Aib, Cba, Cit, D-Trp, D-Tza, Lys, Lys(Ac), Leu, Mle, Gln and Thp;
X10 is an amino acid residue selected among a residue of Asp, Glu, Orn and Lys;
X11 is an amino acid residue selected among a residue of Asn and Ala;
X12 may be absent or is an amino acid residue selected among a residue of Asn and Ala;
R² is —NH₂ or —OH,
or a salt or solvate thereof; and
wherein a lactam bond between X1 and X10 is formed.

A peptide compound of formula (I) inhibits the binding of interleukin-23 (IL-23) to an interleukin-23-receptor (IL-23R).

In some embodiments, a salt or a solvate of a peptide compound of the disclosure may be a pharmaceutically acceptable salt or solvate.

As shown in the Example section, in some embodiments the peptide compounds of formula (I) have a good resistance to pH as low as 1.2, with less than 30% of purity loss, and with a resistance extending in a range of pH going from 1.2 to 7.4. Such capabilities make the peptide compounds of the disclosure usable per oral route, where in the digestive tractus the pH may range from about 7.4, in the mouth, to about 1.2, in the stomach. Further some peptide compounds present good stability in simulated gastric and intestinal fluids. Finally, in some embodiments the peptide compounds of the disclosure have the capacity to inhibit the binding of IL-23 to IL-23R with IC₅₀ in the nanomolar range below about 100 nM, or below about 50 nM, or below about 20 nM, or in the range of from about 0.1 nM to about 50 nM.

In some embodiments, a peptide compound of formula (I) has a sequence selected among SEQ ID NO: 1 to 51, or a salt or solvate thereof.

In some embodiments, a peptide compound of formula (I) has a sequence selected among SEQ ID NO: 1-21 and 23-51, or a salt or solvate thereof.

In some embodiments, a peptide compound of formula (I) has a sequence selected among:
SEQ ID NO: 1-10, 12-18, 23-25, 28-35, 37-41 and 43-51; or
SEQ ID NO: 1-10,13-17, 24-25, 28-35, 37, 39-41 and 43-51; or
SEQ ID NO: 1-10, 14-16, 24, 29, 32-35, 37, 39-41, 43-49 and 51;
SEQ ID NO: 1-10, 15, 24, 32-35, 37, 39-41, 43-49 and 51; or
SEQ ID NO: 1-10, 15, 24, 32-34, 37, 39-41, 43, 45-49 and 51; or
SEQ ID NO: 1-8, 15, 24, 32, 34, 39, 41, 43, 45-48 and 51; or
SEQ ID NO: 1-6, 15, 24, 39, 41, 43, 45-47 and 51; or
SEQ ID NO: 1-2, 45 and 51;
or a salt or solvate thereof.

In some embodiments, the disclosure relates to a peptide compound of formula (I) for use for treating and/or preventing an autoimmune or an inflammatory disease in a subject in need thereof.

In some embodiments, the autoimmune or inflammatory disease is selected from inflammatory bowel disease, such as Crohn's disease and ulcerative colitis, psoriasis, psoriatic arthritis and hidradenitis suppurativa.

In some embodiments, the disclosure relates to a pharmaceutical composition comprising at least one peptide compound of formula (I) of the disclosure, or a pharmaceutically acceptable salt or solvate thereof, and at least one pharmaceutically acceptable excipient.

In some embodiments, the disclosure relates to a use of a peptide compound of formula (I) of the disclosure, for the manufacture of a medicament for treating an autoimmune or an inflammatory disease.

In some embodiments, the disclosure relates to a method for treating and/or preventing an autoimmune or an inflammatory disease in a subject in need thereof, the method comprising administering to said subject a peptide compound according to the disclosure.

### DETAILED DESCRIPTION

### Definitions

Unless otherwise defined herein, scientific and technical terms used in connection with the present disclosure shall have the meanings that are commonly understood by those of ordinary skill in the art. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, may provide one of skill with a general dictionary of many of the terms used in this disclosure. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, virology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular.

Units, prefixes, and symbols are denoted in their International Units System accepted form. Numeric ranges are inclusive of the numbers defining the range. Unless otherwise indicated, amino acid sequences are written left to right in amino to carboxy orientation. The headings provided herein are not limitations of the various aspects of the disclosure. Accordingly, the terms defined immediately below are more fully defined by reference to the specification in its entirety.

Although a number of documents are cited herein, this citation does not constitute an admission that any of these documents forms part of the common general knowledge in the art.

Throughout this specification and embodiments, the words “have” and “comprise,” or variations such as “has,” “having,” “comprises,” or “comprising” are used interchangeably and have the same meaning as comprising.

It is to be noted that the term “a” or “an” means one or more; for example, “a peptide compound,” is understood to mean one or more peptide compounds. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, o″ C; A or C; A “r B; B or C; A and C; A and B; B and C; A (alone); B (alone); and C (alone).

The term “approximately” or “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or ±0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

Peptide compound. The term “peptide compound” encompasses a singular “peptide compound” as well as plural “peptide compounds,” and refers to a molecule comprising amino acids linked by amide bonds. A peptide compound of the disclosure comprises natural and unnatural amino acids. A peptide of the disclosure comprises amide and alkyl bonds based on natural and non-natural amino acids engaged in the bond. A peptide compound of the disclosure is cyclized. A peptide compound may comprise additional modifications with functionalized amino acid.

Amino acids are referred to herein by either their name, their commonly known three letter symbols or by the one-letter symbols recommended by the IUPAC-IUB Biochemical Nomenclature Commission. Therefore, the amino acid sequences of the compounds described herein contain the conventional one letter and three letter codes for naturally occurring amino acids, as well as generally accepted three letter codes for other non-natural amino acids, such as Aib for α-aminoisobutyric acid, Orn for ornithine or Ahx for α-aminohexanoic acid.

The term “amino acid” or “any amino acid” as used here refers to any and all amino acids, including naturally occurring amino acids, modified amino acids, and non-natural amino acids. It includes both Dand L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. Except otherwise indicated, L-amino acids are used throughout the disclosure. D-amino acids are indicated as such.

Residue. Within the disclosure, the term “residue” intends to refer to a moiety within a peptide compound, which results from the binding of an amino acid to another amino acid. Residue is what remains of an amino acid after the removal of the elements of water.

Amide bond. The expression “amide bond” refers to a bond obtained via the coupling between an amine moiety of an amino acid of a peptide compound of the disclosure with a carboxylic acid moiety of another amino acid of this peptide compound. For example, an amide bond may be obtained between a carboxylic moiety of X1 being Asp, D-Asp, Glu, D-Glu, Lys, D-Lys, Orn, D-Orn, Ahx or Apt and the amine moiety of X2 being Asn, D-Asn, Arg or Gln.

Lactam bond. The expression “lactam bond” refers to a cyclic amide bond (—C(═O)—NH—) obtained via the coupling between an amine moiety (—NH₂) of an amino acid of a peptide compound of the disclosure with a carboxylic acid moiety (—COOH) of another amino acid of this peptide compound. The amine and carboxylic acid moieties forming the lactam bond are from the side chains of the amino acids, when the side chain comprises an amino or a carboxylic group. Alternatively, a lactam bond may be formed between the amine moiety of Ahx or Apt and the carboxylic moiety of the side chain of X10 being Asp or Glu.

For example, a lactam bond may be obtained between the amine moiety of the side chain of X1 being Lys, D-Lys, Orn or D-orn, and the carboxylic moiety of the side of chain of X10 being Asp or Glu. Alternatively, a lactam bond may be obtained between the carboxylic moiety of the side chain of X1 being Asp, D-Asp, Glu or D-Glu and the amine moiety of the side of chain of X10 being Orn or Lys.

Activity. The term “activity” used with regard to a peptide compound refers to the capability of the peptide compound to inhibit the interleukin-23 receptor. The term “activity” as used herein also refers to the capability of a peptide compound to inhibit the intracellular STAT3 phosphorylation induced by activation of the IL-23 receptor.

Potency. “Potency” is a measure of the ability of a peptide compound to inhibit the interleukin-23 receptors activation measured in a cell-based assay, as for example presented in the Examples. Numerically, it may be expressed as the “IC₅₀” value, which is the effective concentration of a compound that inhibits a half maximal increase of response (e.g., phosphorylation of STAT3 induced by the IL-23 receptor activation) in a dose-response experiment.

Peptide compounds of the disclosure have interleukin-23 receptor affinity. This term refers to the ability to bind to the interleukin-23 receptor t. Peptide compounds of the disclosure can be tested for interleukn-23 receptor affinity or activity using the assays described in Methods and results shown in Table 6 herein.

“To inhibit the binding of interleukin-23 to an interleukin-23-receptor”. The expression “to inhibit the binding of interleukin-23 to an interleukin-23-receptor” refers to a property of a peptide compound of the disclosure to prevent or reduce the binding of the IL-23 receptor by its ligand IL-23, so as to prevent or reduce activation of a downstream cellular response induced by the binding of IL-23 to its receptor. The capacity of a peptide compound to inhibit the binding of IL-23 to IL-23R is measured by assays disclosed in the Example section. This property is measured as a binding affinity and can be expressed as the “IC₅₀” value, which is the effective concentration of a compound that induces a half maximal decrease of the binding of IL-2.3 to its receptor in a dose-response experiment. In some embodiments, a compound of the disclosure has a binding affinity to the interleukin 23 receptor of 100 nM or below (i.e., IC_{50≤100} nM).

The expressions “pharmaceutically acceptable solvate” and “pharmaceutically acceptable salt” refers to solvates or salts of peptide compounds of the disclosure which can be used in the formulation of a pharmaceutical composition, and which are physiologically acceptable, i.e., safe and effective for use in mammals. A pharmaceutically acceptable salt or solvate may be suitable for oral administration.

“Administer” or “administering,” as used herein refers to delivering to a subject a peptide compound or a composition described herein. The peptide compound or 5 the composition can be administered to a subject using methods known in the art. In particular, the peptide compound or composition can be administered orally, sublingually, buccally, nasally, rectally, vaginally or via pulmonary route, or again intravenously, subcutaneously, intramuscularly, or intradermally. In some embodiments, the administration is an oral administration.

Table 1 presents the codes used for amino acids used herein:

TABLE 1
Natural and non-natural amino acids

| Abbreviation | Name | Structure |
|---|---|---|
| Agb | (2S)-2-amino-5- guanidino-butyric acid | 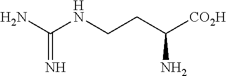 [SMILES: N=C(N)NCC[C@H](N)C(=O)O] |
| Ahx | 6-aminohexanoic acid | 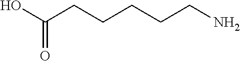 |
| Aib | 2-Aminoisobutyric acid | 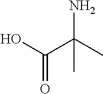 |
| Ala (or A) | L-alanine 2-Aminopropanoic acid | 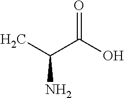 |
| Apt | 5-aminohpentanoic acid | 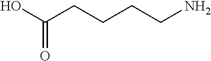 |
| Arg (or R) | L-Arginine 2-Amino-5- guanidinopentanoic acid | 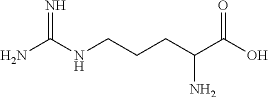 [SMILES: N=C(N)NCCCC(N)C(=O)O] |
| Asn (or N) | L-asparagine 2-Amino-3- carbamoylpropanoic acid | 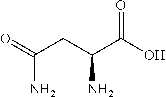 |
| Asp (or D) | L-aspartic acid 2-aminobutanedioic acid | 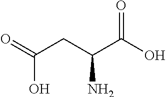 |
| Cba | B-cyclobutyl-L-alanine | 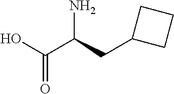 |
| Cit | L-citrulline | 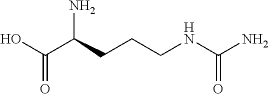 [SMILES: NC(=O)NCCC[C@H](N)C(=O)O] |
| Gln (or Q) | L-Glutamine 2,5-diamino-5- oxopentanoic acid | 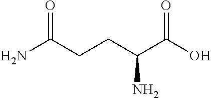 |
| Glu (or E) | L-Glutamic acid 2-Aminopentanedioic acid | 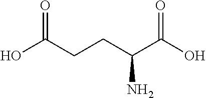 |
| His (or H) | L-Histidine 2-Amino-3- (1H-imidazol- 4-yl)propanoic acid | 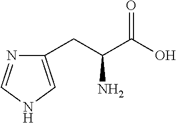 [SMILES: N[C@@H](Cc1c[nH]cn1)C(=O)O] |
| Leu (or L) | L-Leucine 2-Amino-4- methylpentanoic acid | 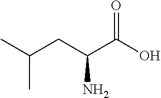 |
| Lys (or K) | L-Lysine 2,6-diaminohexanoic acid | 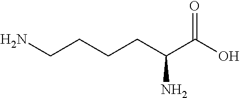 |
| Lys(Ac) (or K(Ac)) | ε-acetyl-L-lysine | 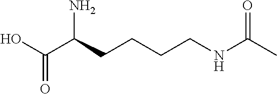 |
| Mle | α-methyl-L-leucine | 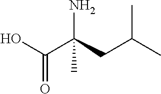 |
| Nak | 1-naphthyl-L-alanine | 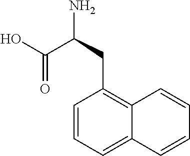 [SMILES: N[C@@H](Cc1cccc2ccccc12)C(=O)O] |
| Nal | 2-naphthyl-L-alanine | 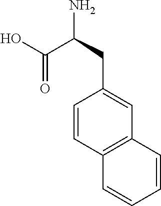 [SMILES: N[C@@H](Cc1ccc2ccccc2c1)C(=O)O] |
| Orn | Ornithine | 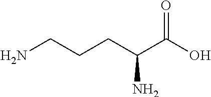 |
| Thp | 4-amino- tetrahydropyran-4- carboxylic acid | 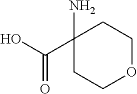 |
| Thr (or T) | L-threonine 2-amino-3- hydroxybutanoic acid | 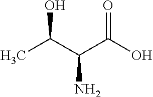 |
| Trp (or W) | L-tryptophan (2S)-2-amino-3-(1H- indol-3-yl) propanoic acid | 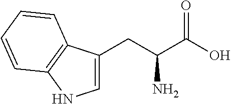 [SMILES: N[C@@H](Cc1c[nH]c2ccccc12)C(=O)O] |
| Tyr (or Y) | L-Tyrosine 2-amino-3-(4- hydroxyphenyl) propanoic | 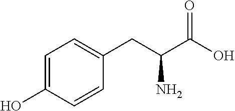 [SMILES: N[C@@H](Cc1ccc(O)cc1)C(=O)O] |
| Tza | B-(4-thiazolyl)-alanine (L-4-Thiazolylalanine) | 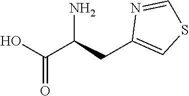 [SMILES: N[C@@H](Cc1cscn1)C(=O)O] |
| Val (or V) | L-Valine 2-Amino-3- methylbutanoic acid | 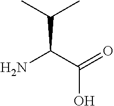 |
| Wcl | 5-chloro-L-tryptophan | 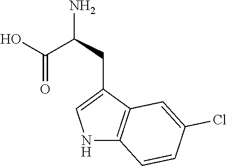 [SMILES: N[C@@H](Cc1c[nH]c2ccc(Cl)cc12)C(=O)O] |
| Wfl | 5-fluoro-L-tryptophan | 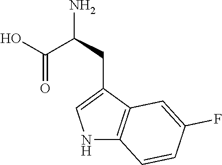 [SMILES: N[C@@H](Cc1c[nH]c2ccc(F)cc12)C(=O)O] |
| Wme | 1-methyl-L-tryptophan | 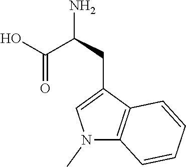 [SMILES: Cn1cc(C[C@H](N)C(=O)O)c2ccccc21] |
| Yae | 4-aminoethoxy-L- phenylalanine | 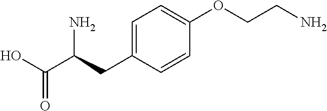 [SMILES: NCCOc1ccc(C[C@H](N)C(=O)O)cc1] |
| Yme | 4-methoxy-L- phenylalanine | 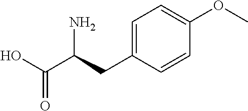 |

It is appreciated that certain features of the disclosure which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this disclosure belongs. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients include combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Peptide Compounds

The disclosure relates to a peptide compound binding to IL-23 receptor (IL23-R) and inhibiting or reducing the binding of interleukin-23 (IL-23) to its receptor.

A peptide compound of the disclosure is a peptide compound of formula (I):

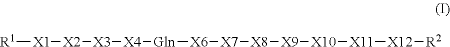

wherein
R¹ is —H or acetyl;
X1 is an amino acid residue selected among a residue of Asp, D-Asp, Glu, D-Glu, Lys, D-Lys, Orn, D-Orn, Ahx and Apt;
X2 is an amino acid residue selected among a residue of Asn, D-Asn, Arg and Gln;
X3 is an amino acid residue selected among a residue of His, Lys, Arg, Asn,
Gln, Trp and Thr;
X4 is an amino acid residue selected among a residue of Trp, Wme, Nak, Wcl and Wfl;
X6 is an amino acid residue selected among a residue of Arg, Leu, Ala, Val, Glu, Aib and Agb;
X7 is an amino acid residue selected among a residue of Yme, Tyr, Trp and Yae;
X8 is an amino acid residue selected among a residue of Nal, Nak and Trp;
X9 is an amino acid residue selected among a residue of Arg, Aib, Cba, Cit, D-Trp, D-Tza, Lys, Lys(Ac), Leu, Mle, Gln and Thp;
X10 is an amino acid residue selected among a residue of Asp, Glu, Orn and Lys;
X11 is an amino acid residue selected among a residue of Asn and Ala;
X12 may be absent or is an amino acid residue selected among a residue of Asn and Ala;
R² is —NH₂ or —OH,
or a salt or solvate thereof; and
wherein a lactam bond between X1 and X10 is formed.

A peptide compound of formula (I) inhibits the binding of interleukin-23 (IL-23) to an interleukin-23-receptor (IL-23R).

In some embodiments, a salt or a solvate of a peptide compound of the disclosure may be a pharmaceutically acceptable salt or solvate.

The term “solvate” when used with regard to a peptide compound of the disclosure refers to an aggregate consisting of a peptide compound with one or more solvent molecules, e.g., organic solvent molecules and/or water. The source of such solvate can be from the solvent of crystallization, inherent in the solvent of preparation or crystallization, or adventitious to such solvent. Such solvates are within the scope of the present disclosure.

The term “salt” when used with respect to a peptide compound of the disclosure intends to refer to an ionic form of a peptide compound of the disclosure, negatively or positively charged, in ionic interaction with positively charged cations or negatively charged anions. Such salts include acid or base addition salts formed with inorganic acids or with organic acids or bases.

The side chain of X1 and the side chain of X10 form together a lactam bond. The carboxylic or amine moiety of the side chain of the amino acid residue represented by X1 and the amine or carboxylic moiety of the side chain of the amino acid residue represented by X10 form together lactam bond.

In the following of the disclosure, the symbol “@1” represents a lactam bond joining two amino acid residues. For example, in the following SEQ ID NO.: 1: D-Lys(Ac)(@ 1)-Asn-Thr-Trp-Gln-Leu-Yme-Nal-Aib-Asp(@1)-Asn-Asn-NH₂, the symbols “@1” represent the lactam bond joining D-Lys(Ac) in position 1 and Asp in position 10.

In some embodiments, peptide compounds of formula (I) comprise a linear backbone of amino carboxylic acids linked by peptide, such as carboxamide bonds. The amino carboxylic acids are α-amino carboxylic acids and, unless otherwise indicated, L-α-amino carboxylic acids (as for example D-Lysine (D-Lys).

Peptide compounds of formula (I) comprise functionalized amino acids, as for example N-methylated amino acids, such as N-Me-L-Tyrosine (N-MeTyr).

Peptide compounds of formula (I) comprise amino acids with amino groups in the side chain wherein one hydrogen atom is substituted by an acetyl residue, which is indicated as for example Lys(Ac).

Peptide compounds of formula (I) comprise amino acids in X1 wherein one hydrogen atom of the N-terminal amino group is substituted by an acetyl residue, which is indicated as for example Lys(Ac)(@1).

Peptide compounds of formula (I) comprise an amino acid in X1 which is not an alpha-amino acid, such as Ahx and Apt, wherein said amino acid in X1 is used to form a lactam bond with the amino acid in X10.

Amino acids residues within the peptide compound of formula (I) are numbered consecutively from 1 to 12 in the conventional N-terminal to C-terminal direction. Reference to a “position” within a peptide compound of formula (I) should be constructed accordingly.

The disclosure is further described by the following embodiments.

In some embodiments of peptide compounds of formula (I), R¹ is —H or acetyl.

In some embodiments of peptide compounds of formula (I), R¹ is —H.

In some embodiments of peptide compounds of formula (I), R¹ is acetyl.

In some embodiments of peptide compounds of formula (I), when X1 is Ahx or Apt, R¹ is —H.

In some embodiments of peptide compounds of formula (I), R² is —NH₂ or —OH.

In some embodiments of peptide compounds of formula (I), R² is —NH₂.

In some embodiments of peptide compounds of formula (I), X1 is an amino acid residue selected among a residue of Asp, D-Asp, Glu, Lys, D-Lys, Orn, D-Orn, Ahx and Apt.

In some embodiments of peptide compounds of formula (I), X1 is a residue of Asp.

In some embodiments of peptide compounds of formula (I), X1 is a residue of D-Asp.

In some embodiments of peptide compounds of formula (I), X1 is a residue of Glu.

In some embodiments of peptide compounds of formula (I), X1 is a residue of Lys.

In some embodiments of peptide compounds of formula (I), X1 is a residue of D-Lys.

In some embodiments of peptide compounds of formula (I), X1 is a residue of Orn.

In some embodiments of peptide compounds of formula (I), X1 is a residue of D-Orn.

In some embodiments of peptide compounds of formula (I), X1 is a residue of Ahx.

In some embodiments of peptide compounds of formula (I), X1 is a residue of Apt.

In some embodiments of peptide compounds of formula (I), X2 is an amino acid residue selected among a residue of Asn and Arg.

In some embodiments of peptide compounds of formula (I), X2 is a residue of Asn.

In some embodiments of peptide compounds of formula (I), X2 is a residue of Arg.

In some embodiments of peptide compounds of formula (I), X3 is an amino acid residue selected among a residue of His, Arg and Thr.

In some embodiments of peptide compounds of formula (I), X3 is a residue of His.

In some embodiments of peptide compounds of formula (I), X3 is a residue of Arg.

In some embodiments of peptide compounds of formula (I), X3 is a residue of Trp.

In some embodiments of peptide compounds of formula (I), X4 is an amino acid residue selected among a residue of Trp and Wme.

In some embodiments of peptide compounds of formula (I), X4 is a residue of Trp.

In some embodiments of peptide compounds of formula (I), X4 is a residue of Wme.

In some embodiments of peptide compounds of formula (I), X6 is an amino acid residue selected among a residue of Arg, Leu, Ala, Val, Aib and Agb,

In some embodiments of peptide compounds of formula (I), X6 is an amino acid residue selected among a residue of Leu, Ala, Val and Aib.

In some embodiments of peptide compounds of formula (I), X6 is an amino acid residue selected among a residue of Leu, Ala and Arg.

In some embodiments of peptide compounds of formula (I), X6 is a residue of Arg.

In some embodiments of peptide compounds of formula (I), X6 is a residue of Leu.

In some embodiments of peptide compounds of formula (I), X6 is a residue of Ala.

In some embodiments of peptide compounds of formula (I), X6 is a residue of Val.

In some embodiments of peptide compounds of formula (I), X6 is a residue of Aib.

In some embodiments of peptide compounds of formula (I), X6 is a residue of Agb.

In some embodiments of peptide compounds of formula (I), X7 is an amino acid residue selected among a residue of Yme, Tyr, and Yae.

In some embodiments of peptide compounds of formula (I), X7 is a residue of Yme.

In some embodiments of peptide compounds of formula (I), X7 is a residue of, Tyr.

In some embodiments of peptide compounds of formula (I), X7 is a residue of Yae.

In some embodiments of peptide compounds of formula (I), X7 is an amino acid residue selected among a residue of Tyr and Yme.

In some embodiments of peptide compounds of formula (I), X8 is an amino acid residue selected among a residue of Nal, Nak and Trp.

In some embodiments of peptide compounds of formula (I), X8 is an amino acid residue selected among a residue of Nal and Nak.

In some embodiments of peptide compounds of formula (I), X8 is a residue of Nal.

In some embodiments of peptide compounds of formula (I), X8 is a residue of Nak.

In some embodiments of peptide compounds of formula (I), X8 is a residue of Trp.

In some embodiments of peptide compounds of formula (I), X9 is an amino acid residue selected among a residue of Aib, Leu, Arg, Thp, Cit, D-Tza, Lys(Ac), Mle, and Gln.

In some embodiments of peptide compounds of formula (I), X9 is a residue of Aib.

In some embodiments of peptide compounds of formula (I), X9 is a residue of Leu.

In some embodiments of peptide compounds of formula (I), X9 is a residue of Arg.

In some embodiments of peptide compounds of formula (I), X9 is a residue of Cit.

In some embodiments of peptide compounds of formula (I), X9 is a residue of D-Tza.

In some embodiments of peptide compounds of formula (I), X9 is a residue of Lys(Ac).

In some embodiments of peptide compounds of formula (I), X9 is a residue of Mle.

In some embodiments of peptide compounds of formula (I), X9 is a residue of Gln.

In some embodiments of peptide compounds of formula (I), X9 is an amino acid residue selected among a residue of Aib and Lys(Ac).

In some embodiments, X1 is an amino acid residue selected among a residue of Ahx, Apt, Lys, D-Lys, Orn, and D-Orn, and X10 is an amino acid residue selected among a residue of Asp, Glu, D-Asp and D-Glu.

In some embodiments, X1 is an amino acid residue selected among a residue of Ahx, Apt, Lys, D-Lys, Orn, and D-Orn, and X10 is an amino acid residue selected among a residue of Asp and Glu.

In some embodiments, X1 is an amino acid residue selected among a residue of Asp, Glu, D-Asp and D-Glu, and X10 is an amino acid residue selected among a residue of Lys, D-Lys, Orn and D-Orn.

In some embodiments, X1 is an amino acid residue selected among a residue of Asp, Glu, D-Asp and D-Glu, and X10 is an amino acid residue selected among a residue of Lys and Orn.

In some embodiments, X1 is an amino acid residue selected among a residue of Asp, D-Asp and Glu, and X10 is an amino acid residue selected among a residue of Lys, Orn and D-Orn.

In some embodiments, X1 is an amino acid residue selected among a residue of Asp, D-Asp and Glu, and X10 is an amino acid residue selected among a residue of Lys and Orn.

In some embodiments, X1 is an amino acid residue selected among a residue of Ahx, Apt, Lys, D-Lys, Orn and D-Orn, and X10 is an amino acid residue selected among a residue of Asp and Glu.

In some embodiments:
X1 is an amino acid residue selected among a residue of Asp, D-Asp and Glu, and X10 is an amino acid residue selected among a residue of Lys, Orn and D-Orn or
X1 is an amino acid residue selected among a residue of Ahx, Apt, Lys, D-Lys, Orn and D-Orn, and X10 is an amino acid residue selected among a residue of Asp and Glu.

In some embodiments, X1 is an amino acid residue selected among a residue of Ahx and Apt, and R¹ is —H.

### First Group of Compounds

In some embodiments, peptide compounds of formula (I) comprise a first group of compounds wherein:
R¹ is —H or acetyl;
X1 is an amino acid residue selected among a residue of Asp, D-Asp, Glu, Lys, D-Lys, Orn, D-Orn, Ahx and Apt;
X2 is an amino acid residue selected among a residue of Asn and Arg;
X3 is an amino acid residue selected among a residue of His, Arg and Thr;
X4 is an amino acid residue selected among a residue of Trp and Wme;
X6 is an amino acid residue selected among a residue of Arg, Leu, Ala, Val, Aib and Agb;
X7 is an amino acid residue selected among a residue of Yme, Tyr and Yae;
X8 is an amino acid residue selected among a residue of Nal, Nak and Trp;
X9 is an amino acid residue selected among a residue of Aib, Leu, Arg, Thp, Cit, D-Tza, Lys(Ac), Mle and Gln;
X10 is an amino acid residue selected among a residue of Asp, Glu, Orn and Lys;
X11 is an amino acid residue selected among a residue of Asn and Ala;
X12 may be absent or is an amino acid residue selected among a residue of Asn and Ala; and
R² is —NH₂.

In some embodiments, peptide compounds of formula (I) comprise a first group of compounds wherein:
X1 is an amino acid residue selected among a residue of Asp, D-Asp, Glu, Lys, D-Lys, Orn, D-Orn, Ahx and Apt;
X2 is an amino acid residue selected among a residue of Asn and Arg;
X3 is an amino acid residue selected among a residue of His, Arg and Thr;
X4 is an amino acid residue selected among a residue of Trp and Wme;
X6 is an amino acid residue selected among a residue of Arg, Leu, Ala, Val, Aib and Agb;
X7 is an amino acid residue selected among a residue of Yme, Tyr and Yae;
X9 is an amino acid residue selected among a residue of Aib, Leu, Arg, Thp, Cit, D-Tza, Lys(Ac), Mle and Gln;
and
R² is —NH₂.

In some embodiments of the first group of compounds:
X1 is an amino acid residue selected among a residue of Asp, D-Asp and Glu, and X10 is an amino acid residue selected among a residue of Lys, Orn and D-Orn
or
X1 is an amino acid residue selected among a residue of Ahx, Apt, Lys, D-Lys, Orn and D-Orn, and X10 is an amino acid residue selected among a residue of Asp and Glu.

In some embodiments of the first group of compounds, X1 is an amino acid residue selected among a residue of Ahx and Apt, and R¹ is —H.

In some embodiments of the first group of compounds, X1 is an amino acid residue selected among a residue of Asp, D-Asp, Glu, Lys, D-Lys, Orn and D-Orn and R¹ is acetyl.

In some embodiments of the first group of compounds, R¹ is —H or acetyl.

In some embodiments of the first group of compounds, R¹ is acetyl.

In some embodiments of the first group of compounds, a peptide compound of formula (I) may be such that:
X1 is a residue of D-Asp, and
X10 is a residue of Lys,
wherein the side chain carboxy group of X1 forms a lactam bond with the side chain amino group of X10.

In some embodiments of the first group of compounds:
X1 is a residue of D-Asp, and
X10 is a residue of Orn,
wherein the side chain carboxy group of X1 forms a lactam bond with the side chain amino group of X10.

In some embodiments of the first group of compounds:
X1 is a residue of Asp, and
X10 is a residue of Orn,
wherein the side chain carboxy group of X1 forms a lactam bond with the side chain amino group of X10.

In some embodiments of the first group of compounds:
X1 is a residue of Glu, and
X10 is a residue of Orn,
wherein the side chain carboxy group of X1 forms a lactam bond with the side chain amino group of X10.

In some embodiments of the first group of compounds:
X1 is a residue of Glu, and
X10 is a residue of Orn,
wherein the side chain carboxy group of X1 forms a lactam bond with the side chain amino group of X10.

In some embodiments of the first group of compounds:
X1 is a residue of D-Lys and
X10 is a residue of D-Glu,
wherein the side chain amino group of X1 forms a lactam bond with the side chain carboxy group of X10.

In some embodiments of the first group of compounds:
X1 is a residue of D-Lys and
X10 is a residue of Asp,
wherein the side chain amino group of X1 forms a lactam bond with the side chain carboxy group of X10.

In some embodiments of the first group of compounds:
X1 is a residue of D-Lys and
X10 is a residue of Glu,
wherein the side chain amino group of X1 forms a lactam bond with the side chain carboxy group of X10.

In some embodiments of the first group of compounds:
X1 is a residue of Lys and
X10 is a residue of Asp,
wherein the side chain amino group of X1 forms a lactam bond with the side chain carboxy group of X10.

In some embodiments of the first group of compounds:
X1 is a residue of D-Orn and
X10 is a residue of Asp,
wherein the side chain amino group of X1 forms a lactam bond with the side chain carboxy group of X10.

In some embodiments of the first group of compounds:
X1 is a residue of Orn and
X10 is a residue of Glu,
wherein the side chain amino group of X1 forms a lactam bond with the side chain carboxy group of X10.

In some embodiments of the first group of compounds:
X1 is a residue of Ahx and
X10 is a residue of Glu,
wherein the amino group of X1 forms a lactam bond with the side chain carboxy group of X10.

In some embodiments of the first group of compounds:
X1 is a residue of Ahx and
X10 is a residue of Asp,
wherein the amino group of X1 forms a lactam bond with the side chain carboxy group of X10.

In some embodiments of the first group of compounds:
X1 is a residue of Apt and
X10 is a residue of Asp,
wherein the side chain amino group of X1 forms a lactam bond with the side chain carboxy group of X10.

In some embodiments of the first group of compounds, when X1 is a residue of D-Asp, then X10 is a residue of Lys or Orn.

In some embodiments of the first group of compounds, when X1 is a residue of Asp or Glu, then X10 is a residue of Orn.

In some embodiments of the first group of compounds, when X1 is a residue of D-Lys, D-Orn or Ahx, then X10 is a residue of Asp or Glu.

In some embodiments of the first group of compounds, when X1 is a residue of Lys or Apt, then X10 is a residue of Asp.

In some embodiments of the first group of compounds, when X1 is a residue of D-Orn, then X8 is a residue of Nal or Nak, and/or X10 is a residue of Asp or Glu.

### Second Group of Compounds

In some embodiments, peptide compounds of formula (I) comprise a second group of compounds wherein:
X1 is an amino acid residue selected among a residue of Asp, D-Asp, Glu, Lys, D-Lys, Orn, Ahx and Apt;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu, Ala, Val, Aib and Agb;
X7 is an amino acid residue selected among a residue of Yme, Tyr and Yae;
X8 is an amino acid residue selected among a residue of Nal and Nak;
X9 is an amino acid residue selected among a residue of Aib, Leu, Arg, Thp, Cit, D-Tza, Lys(Ac), Mle, and Gln;
X10 is an amino acid residue selected among a residue of Asp, Glu, Orn and Lys;
X11 is an amino acid residue selected among a residue of Asn and Ala;
X12 is an amino acid residue selected among a residue of Asn and Ala.

In some embodiments of the second group of compounds, R¹ is —H or acetyl.

In some embodiments of the second group of compounds, R¹ is acetyl.

In some embodiments of the second group of compounds, peptide compounds of formula (I) inhibit the binding of IL-23 to IL-23 receptor with an IC₅₀ of about 20 nM or less, and for example of about 15 nM or less, and for example in the range of about 1 nM to about 20 nM.

### Third Group of Compounds

In some embodiments, peptide compounds of formula (I) comprise a third group of compounds wherein:
X1 is an amino acid residue selected among a residue of Asp, D-Asp, Glu, Lys, D-Lys, Ahx and Apt;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu, Ala, Val, Aib and Agb;
X7 is an amino acid residue selected among a residue of Yme and Tyr;
X8 is an amino acid residue selected among a residue of Nal and Nak;
X9 is an amino acid residue selected among a residue of Aib, Leu, Arg, Thp, Cit, D-Tza, Lys(Ac), Mle and Gln;
X10 is an amino acid residue selected among a residue of Asp, Glu and Orn;
X11 is an amino acid residue selected among a residue of Asn and Ala;
X12 is an amino acid residue selected among a residue of Asn and Ala.

In some embodiments of the third group of compounds, R¹ is —H or acetyl. In some embodiments of the third group of compounds, R¹ is acetyl.

In some embodiments of the third group, peptide compounds of formula (I) inhibit the binding of IL-23 to IL-23 receptor with an IC₅₀ of about 10 nM or less.

In some embodiments of the third group, peptide compounds of formula (I) inhibit the binding of IL-23 to IL-23 receptor with an IC₅₀ in the range of about 0.5 nM to about 10 nM.

### Fourth Group of Compounds

In some embodiments, peptide compounds of formula (I) comprise a fourth group of compounds wherein:
X1 is an amino acid residue selected among a residue of Asp, Glu, Lys, D-Lys, Ahx and Apt;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu, Ala, Aib and Agb;
X7 is an amino acid residue selected among a residue of Yme and Tyr;
X8 is an amino acid residue selected among a residue of Nal and Nak;
X9 is an amino acid residue selected among a residue of Aib, Leu, Arg, Thp, Cit, Lys(Ac) and Mle;
X10 is an amino acid residue selected among a residue of Asp, Glu and Orn;
X11 is an amino acid residue selected among a residue of Asn and Ala;
X12 is an amino acid residue selected among a residue of Asn and Ala.

In some embodiments of the fourth group of compounds, R¹ is —H or acetyl.

In some embodiments of the fourth group of compounds, R¹ is acetyl.

In some embodiments of the fourth group, peptide compounds of formula (I) inhibit the binding of IL-23 to IL-23 receptor with an IC₅₀ of about 6 nM or less.

In some embodiments of the fourth group, peptide compounds of formula (I) inhibit the binding of IL-23 to IL-23 receptor with an IC₅₀ in the range of about 0.5 nM to about 6 nM.

### Fifth Group of Compounds

In some embodiments, peptide compounds of formula (I) comprise a fifth group of compounds wherein:
X1 is an amino acid residue selected among a residue of Asp, Glu, Lys, D-Lys, Ahx and Apt;
X2 is an amino acid residue selected among a residue of Asn and Arg;
X3 is an amino acid residue selected among a residue of Arg, and Thr;
X4 is an amino acid residue selected among a residue of Trp and Wme;
X6 is an amino acid residue selected among a residue of Arg, Leu, Ala, Aib and Agb;
X7 is an amino acid residue selected among a residue of Yme and Tyr;
X8 is an amino acid residue selected among a residue of Nal and Nak;
X9 is an amino acid residue selected among a residue of Aib, Leu, Arg, Thp, Lys(Ac) and Mle;
X10 is an amino acid residue selected among a residue of Asp, Glu, and Orn;
X11 is an amino acid residue selected among a residue of Asn and Ala;
X12 is an amino acid residue selected among a residue of Asn and Ala.

In some embodiments of the fifth group of compounds, R¹ is —H or acetyl.

In some embodiments of the fifth group of compounds, R¹ is acetyl.

In some embodiments of the fifth group, peptide compounds of formula (I) inhibit the binding of IL-23 to IL-23 receptor with an IC₅₀ of about 5 nM or less.

In some embodiments of the fifth group, peptide compounds of formula (I) inhibit the binding of IL-23 to IL-23 receptor with an IC₅₀ in the range of about 0.5 nM to about 5 nM.

### Sixth Group of Compounds

In some embodiments, peptide compounds of formula (I) comprise a sixth group of compounds wherein:
X1 is an amino acid residue selected among a residue of Asp, Lys, D-Lys and Ahx;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu, Ala, Aib and Agb;
X7 is an amino acid residue selected among a residue of Yme and Tyr;
X8 is an amino acid residue selected among a residue of Nal and Nak;
X9 is an amino acid residue selected among a residue of Aib, Leu, Arg, Thp, Lys(Ac) and Mle;
X10 is an amino acid residue selected among a residue of Asp, Glu and Orn;
X11 is an amino acid residue selected among a residue of Asn and Ala;
X12 is an amino acid residue selected among a residue of Asn and Ala.

In some embodiments of the sixth group of compounds, R¹ is —H or acetyl.

In some embodiments of the sixth group of compounds, R¹ is acetyl.

In some embodiments of the sixth group, peptide compounds of formula (I) inhibit the binding of IL-23 to IL-23 receptor with an IC₅₀ of about 4 nM or less.

In some embodiments of the sixth group, peptide compounds of formula (I) inhibit the binding of IL-23 to IL-23 receptor with an IC₅₀ in the range of about 0.5 nM to about 4 nM.

### Seventh Group of Compounds

In some embodiments, peptide compounds of formula (I) comprise a seventh group of compounds wherein:
X1 is an amino acid residue selected among a residue of Asp, Lys, D-Lys and Ahx;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu, Ala and Aib;
X7 is an amino acid residue selected among a residue of Yme and Tyr;
X8 is an amino acid residue selected among a residue of Nal and Nak;
X9 is an amino acid residue selected among a residue of Aib, Arg, Thp and Mle;
X10 is an amino acid residue selected among a residue of Asp, Glu and Orn;
X11 is a residue of Asn;
X12 is an amino acid residue selected among a residue of Asn and Ala.

In some embodiments of the seventh group of compounds, R¹ is —H or acetyl.

In some embodiments of the seventh group of compounds, R¹ is acetyl.

In some embodiments of the seventh group, peptide compounds of formula (I) inhibit the binding of IL-23 to IL-23 receptor with an IC₅₀ of about 3 nM or less.

In some embodiments of the seventh group, peptide compounds of formula (I) inhibit the binding of IL-23 to IL-23 receptor with an IC₅₀ in the range of about 0.5 nM to about 3 nM.

### Eight Group of Compounds

In some embodiments, peptide compounds of formula (I) comprise an eighth group of compounds wherein:
X1 is an amino acid residue selected among a residue of Asp, Lys, D-Lys and Ahx;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Arg, Leu and Ala;
X7 is an amino acid residue selected among a residue of Yme and Tyr;
X8 is a residue of Nal;
X9 is an amino acid residue selected among a residue of Aib, Arg, Thp, and
Mle;
X10 is an amino acid residue selected among a residue of Asp, Glu and Orn;
X11 is a residue of Asn;
X12 is a residue of Asn.

In some embodiments of the eighth group of compounds, R¹ is —H or acetyl.

In some embodiments of the eighth group of compounds, R¹ is acetyl.

In some embodiments of the eighth group, peptide compounds of formula (I) inhibit the binding of IL-23 to IL-23 receptor with an IC₅₀ of about 2 nM or less.

In some embodiments of the eighth group, peptide compounds of formula (I) inhibit the binding of IL-23 to IL-23 receptor with an IC₅₀ in the range of about 0.5 nM to about 2 nM.

### Nineth Group of Compounds

In some embodiments, peptide compounds of formula (I) comprise a nineth group of compounds wherein:
X1 is D-Lys;
X2 is an amino acid residue selected among a residue of Asn and Arg;
X3 is a residue of Thr;
X4 is a residue of Trp;
X6 is an amino acid residue selected among a residue of Leu and Ala;
X7 is a residue of Yme;
X8 is a residue of Nal;
X9 is an amino acid residue selected among a residue of Aib and Thp;
X10 is a residue of Asp;
X11 is a residue of Asn;
X12 is a residue of Asn.

In some embodiments of the nineth group of compounds, R¹ is —H or acetyl.

In some embodiments of the nineth group of compounds, R¹ is acetyl.

In some embodiments of the nineth group, peptide compounds of formula (I) inhibit the binding of IL-23 to IL-23 receptor with an IC₅₀ of about 1 nM or less.

In some embodiments of the nineth group, peptide compounds of formula (I) inhibit the binding of IL-23 to IL-23 receptor with an IC₅₀ in the range of about 0.5 nM to about 1 nM.

### Tenth Group of Compounds

In some embodiments, peptide compounds of formula (I) comprise a tenth group of compounds wherein:
X1 is an amino acid residue selected among a residue of Ahx, Apt, Asp, D-Asp and Glu;
X2 is an amino acid residue selected among a residue of Asn and Arg;
X3 is an amino acid residue selected among a residue of His, Arg and Thr;
X4 is an amino acid residue selected among a residue of Trp and Wme;
X6 is an amino acid residue selected among a residue of Arg, Ala, Aib, Leu, Val and Agb;
X7 is an amino acid residue selected among a residue of Yme, Yae and Tyr;
X8 is a residue of Nal;
X9 is an amino acid residue selected among a residue of Aib, Arg, Thp, Cit, Gln, Lys(Ac), D-Tza, Mle and Leu;
X10 is an amino acid residue selected among a residue of Asp, Orn and Glu;
X11 is an amino acid residue selected among a residue of Asn and Ala;
X12 is absent or is an amino acid residue Asn or Ala.

In some embodiments of the tenth group of compounds, X1 is an amino acid residue selected among a residue of Ahx, Apt, Asp and Glu.

In some embodiments of the tenth group of compounds, X1 is an amino acid residue selected among a residue of Ahx and Asp.

In some embodiments of the tenth group of compounds, X1 is an amino acid residue selected among a residue of Ahx and Apt.

In some embodiments of the tenth group of compounds, R² is —NH₂.

In some embodiments of the tenth group of compounds:
X2 is a residue of Asn,
X3 is a residue of Thr,
X6 is a residue of Agb, Ala or Arg,
X7 is a residue of Yme or Tyr,
X9 is a residue of Aib or Leu,
X10 is a residue of Glu, Asp or Orn,
X11 is a residue of Asn, and
X12 is a residue of Asn.

In some embodiments of the tenth group of compounds
X1 is a residue of Ahx,
X4 is a residue of Trp,
X6 is a residue of Agb or Ala,
X7 is a residue of Yme,
X9 is a residue of Aib, and
X10 is a residue of Glu or Asp.

In some embodiments of the tenth group of compounds,
X1 is a residue of Apt,
X6 is a residue of Ala,
X7 is a residue of Yme,
X9 is a residue of Aib, and
X10 is a residue of Asp.

In some embodiments of the tenth group of compounds,
X1 is a residue of Asp,
X6 is a residue of Arg,
X9 is a residue of Aib or Leu, and
X10 is a residue of Orn.

In some embodiments of the tenth group of compounds,
X1 is a residue of D-Asp,
X4 is a residue of Trp,
X6 is a residue of Arg,
X7 is a residue of Yme,
X9 is a residue of Aib, and
X10 is a residue of Orn.

In some embodiments of the tenth group of compounds,
X1 is a residue of Glu,
X4 is a residue of Wme,
X6 is a residue of Arg,
X7 is a residue of Yme,
X9 is a residue of Aib, and
X10 is a residue of Orn.

In some embodiments of the tenth group of compounds, R¹ is —H or acetyl.

In some embodiments of the tenth group of compounds, R¹ is acetyl.

### Eleventh Group of Compounds

In some embodiments, peptide compounds of formula (I) comprise an eleventh group of compounds wherein:
X1 is an amino acid residue selected among a residue of Ahx and Asp;
X2 is a residue of Asn;
X3 is a residue of Thr;
X4 is a residue of Trp;
X6 is an amino acid residue selected among a residue of Arg, Ala, Aib, Leu and Val;
X7 is an amino acid residue selected among a residue of Yme, Yae and Tyr;
X8 is a residue of Nal;
X9 is an amino acid residue selected among a residue of Aib, Arg, Thp, Cit, Gln, Lys(Ac), Leu, D-Tza, and Mle;
X10 is an amino acid residue selected among a residue of Asp and Orn;
X11 is a residue of Asn; and
X12 is a residue of Asn.

In some embodiments of the eleventh group of compounds, X6 is an amino acid residue of Ala or Arg.

In some embodiments of the eleventh group of compounds, X7 is an amino acid residue of Yme.

In some embodiments of the eleventh group of compounds, X9 is an amino acid residue of Aib.

In some embodiments of the eleventh group, R¹ is —H or acetyl.

In some embodiments of the eleventh group of compounds, R¹ is acetyl.

### Twelfth Group of Compounds

In some embodiments, peptide compounds of formula (I) comprise a twelfth group of compounds wherein:
X1 is an amino acid residue selected among a residue of D-Lys, Orn and Lys;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Aib, Agb, Ala, Arg, Leu and Val;
X7 is an amino acid residue selected among a residue of Yme and Tyr;
X8 is an amino acid residue selected among a residue of Nal and Nak,
X9 is an amino acid residue selected among a residue of Aib, Arg, Leu, Thp, Cit, Gln, Lys(Ac), D-Tza and Mle;
X10 is an amino acid residue selected among a residue of Asp, Orn, Lys and Glu;
X11 is an amino acid residue selected among a residue of Asn and Ala;
X12 is an amino acid residue selected among a residue of Asn and Ala.

In some embodiments, peptide compounds of formula (I) comprise a twelfth group of compounds wherein:
X1 is an amino acid residue selected among a residue of D-Lys, Orn and Lys;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Aib, Agb, Ala, Arg, Leu and Val;
X7 is an amino acid residue selected among a residue of Yme and Tyr;
X8 is a residue of Nal,
X9 is an amino acid residue selected among a residue of Aib, Arg, Leu, Thp, Cit, Gln, Lys(Ac), D-Tza and Mle;
X10 is an amino acid residue selected among a residue of Asp, Orn, Lys and Glu;
X11 is an amino acid residue selected among a residue of Asn and Ala;
X12 is an amino acid residue selected among a residue of Asn and Ala.

In some embodiments of the twelfth group of compounds, R² is —NH₂.

In some embodiments of the twelfth group of compounds, R¹ is —H or acetyl. In some embodiments of the twelfth group of compounds, R¹ is acetyl.

In some embodiments of the twelfth group of compounds, when X6 is a residue of Aib or Val, then X8 is a residue of Nal.

In some embodiments of the twelfth group of compounds, when X4 is a residue of Wme and X6 is a residue of Arg, then X8 is a residue of Nal.

In some embodiments of the twelfth group of compounds, when X9 is a residue of Cit or Gin, then X8 is a residue of Nal.

In some embodiments of the twelfth group of compounds:
X1 is a residue of D-Lys, Orn or Lys;
X2 is a residue of Asn or Arg;
X3 is a residue of Arg or Thr;
X4 is a residue of Trp or Wme;
X6 is a residue of Aib, Agb, Ala, Arg, Val or Leu;
X7 is a residue of Yme or Tyr;
X8 is a residue of Nak or Nal;
X9 is a residue of Aib, Arg, Leu, Cit, Gln, D-Tza, Lys(Ac), Thp or Mle;
X10 is a residue of Asp, Orn, Lys or Glu;
X11 is a residue of Asn or Ala;
X12 is a residue of Asn or Ala; and when X6 is a residue of Aib or Val, then X8 is a residue of Nal, or when
X4 is a residue of Wme and X6 is a residue of Arg, then X8 is a residue of Nal, or when X9 is a residue of Cit or Gln, then and X8 is a residue of Nal.

In some embodiments of the twelfth group of compounds, X1 is an amino acid residue selected among a residue of D-Lys and Lys.

In some embodiments of the twelfth group of compounds, X9 is an amino acid residue selected among a residue of Aib, Arg, Thp, Cit, Gln, Lys(Ac), D-tza and Mle.

In some embodiments of the twelfth group of compounds, X10 is an amino acid residue selected among a residue of Asp and Glu.

In some embodiments of the twelfth group of compounds:
R¹ is acetyl;
X1 is a residue of Lys,
X2 is a residue of Asn;
X3 is a residue of Thr;
X4 is a residue of Trp;
X6 is a residue of Arg;
X7 is a residue of Yme;
X9 is a residue of Aib or Thp;
X10 is a residue of Asp;
X11 is a residue of Asn;
X12 is a residue of Asn.

In some embodiments of the twelfth group of compounds:
R¹ is acetyl;
X1 is a residue of D-Lys or Lys;
X2 is a residue of Asn or Arg;
X3 is a residue of Arg or Thr;
X4 is a residue of Trp or Wme;
X7 is a residue of Yme or Tyr;
X8 is a residue of Nal;
X9 is a residue of Aib, Arg, Thp or Mle;
X10 is a residue of Asp or Glu;
X11 is a residue of Asn;
X12 is a residue of Asn.

In some embodiments of the twelfth group of compounds:
R¹ is acetyl;
X1 is a residue of D-Lys;
X2 is a residue of Asn or Arg;
X3 is a residue of Arg or Thr;
X4 is a residue of Trp or Wme;
X6 is a residue of Ala, Arg or Leu;
X7 is a residue of Yme or Tyr;
X8 is a residue of Nal;
X9 is a residue of Aib, Arg, Thp or Mle;
X10 is a residue of Asp or Glu;
X11 is a residue of Asn;
X12 is a residue of Asn.

In some embodiments of the twelfth group of compounds:
R¹ is acetyl;
X1 is a residue of D-Lys;
X2 is a residue of Asn or Arg;
X3 is a residue of Arg or Thr;
X4 is a residue of Trp or Wme;
X7 is a residue of Yme or Tyr;
X8 is a residue of Nal;
X9 is a residue of Aib, Arg, Thp or Mle;
X10 is a residue of Asp;
X11 is a residue of Asn;
X12 is a residue of Asn.

In some embodiments of the twelfth group of compounds:
R¹ is acetyl;
X1 is a residue of D-Lys;
X2 is a residue of Asn or Arg;
X3 is a residue of Arg or Thr;
X4 is a residue of Trp or Wme;
X6 is a residue of Ala, Arg or Leu;
X7 is a residue of Yme;
X8 is a residue of Nal;
X9 is a residue of Aib, Arg, Thp or Mle;
X10 is a residue of Asp;
X11 is a residue of Asn;
X12 is a residue of Asn.

In some embodiments of the twelfth group of compounds:
R¹ is acetyl;
X1 is a residue of D-Lys;
X2 is a residue of Asn or Arg;
X3 is a residue of Arg or Thr;
X4 is a residue of Trp or Wme;
X7 is a residue of Yme;
X8 is a residue of Nal;
X9 is a residue of Aib or Thp;
X10 is a residue of Asp;
X11 is a residue of Asn;
X12 is a residue of Asn.

### Thirteenth Group of Compounds

In some embodiments, peptide compounds of formula (I) comprise a thirteenth group of compounds wherein:
X1 is an amino acid residue selected among a residue of D-Lys, Orn and Lys;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Aib, Agb, Ala, Arg and Leu;
X7 is an amino acid residue selected among a residue of Yme and Tyr;
X8 is an amino acid residue selected among a residue of Nak and Nal;
X9 is an amino acid residue selected among a residue of Aib, Arg, Lys(Ac), Thp and Mle;
X10 is an amino acid residue selected among a residue of Asp, Orn, Lys and Glu;
X11 is an amino acid residue selected among a residue of Asn and Ala;
X12 is an amino acid residue selected among a residue of Asn and Ala.

In some embodiments of the thirteenth group of compounds, when X6 is a residue of Aib or Arg, then X8 is a residue of Nal.

In some embodiments of the thirteenth group of compounds, when X4 is a residue of Wme and X6 is a residue of Arg, then X8 is a residue of Nal.

In some embodiments of the thirteenth group of compounds, when X11 is a residue of Ala, then X12 is a residue of Asn.

In some embodiments of the thirteenth group of compounds, R² is —NH₂.

In some embodiments of the thirteenth group of compounds, R¹ is H or acetyl.

In some embodiments of the thirteenth group of compounds, R¹ is acetyl.

### Fourteenth Group of Compounds

In some embodiments, peptide compounds of formula (I) comprise a fourteenth group of compounds wherein:
X1 is an amino acid residue selected among a residue of D-Lys and Lys;
X2 is an amino acid residue selected among a residue of Asn and Arg;
X3 is an amino acid residue selected among a residue of Arg and Thr;
X4 is an amino acid residue selected among a residue of Trp and Wme;
X6 is an amino acid residue selected among a residue of Ala, Agb, Arg and Leu;
X7 is an amino acid residue selected among a residue of Yme and Tyr;
X8 is a residue of Nal;
X9 is an amino acid residue selected among a residue of Aib, Arg, Thp and Mle;
X10 is an amino acid residue selected among a residue of Asp and Glu;
X11 is a residue of Asn; and
X12 is a residue of Asn.

In some embodiments of the fourteenth group of compounds, R² is —NH₂.

In some embodiments of the fourteenth group of compounds, R¹ is —H or acetyl.

In some embodiments of the fourteenth group of compounds, R¹ is acetyl.

### Fifteenth Group of Compounds

In some embodiments, peptide compounds of formula (I) comprise a fifteenth group of compounds wherein:
R¹ is —H or acetyl,
X1 is a residue of D-Lys,
X2 is a residue of Asn,
X3 is a residue of Thr,
X4 is a residue of Trp,
X6 is a residue of Aib, Leu or Ala,
X7 is a residue of Yme,
X8 is a residue of Nal or Nak,
X9 is a residue of Aib or Thp,
R² is —NH₂.

In some embodiments of the fifteenth group of compounds
X10 is a residue of Asp,
X11 is a residue of Asn,
X12 is a residue of Asn.

### Sixteenth Group of Compounds

In some embodiments, peptide compounds of formula (I) comprise a sixteenth group of compounds wherein:
R¹ is —H or acetyl,
X1 is a residue of D-Lys,
X2 is a residue of Asn,
X3 is a residue of Thr,
X4 is a residue of Trp,
X6 is a residue of Leu or Ala,
X7 is a residue of Yme,
X8 is a residue of Nal or Nak,
X9 is a residue of Aib,
R² is NH₂.

In some embodiments of the sixteenth group of compounds,
X10 is a residue of Asp,
X11 is a residue of Asn,
X12 is a residue of Asn.

In some embodiments of the sixteenth group of compounds, X8 is a residue of Nal.

### Seventeenth Group of Compounds

In some embodiments, peptide compounds of formula (I) comprise a seventeenth group of compounds wherein:
R¹ is —H or acetyl,
X1 is a residue of D-Lys,
X2 is a residue of Asn,
X3 is a residue of Thr,
X4 is a residue of Trp,
X6 is a residue of Aib,
X7 is a residue of Yme,
X8 is a residue of Nal or Nak,
X9 is a residue of Aib,
R² is —NH₂.

In some embodiments of the seventeenth group of compounds, X1 is a residue of D-Lys.

In some embodiments of the seventeenth group of compounds:
X10 is a residue of Asp,
X11 is a residue of Asn,
X12 is a residue of Asn.

In some embodiments of the seventeenth group of compounds X8 is a residue of Nak.

In some embodiments, a peptide compound of formula (I) has a sequence selected among SEQ ID NO: 1 to 51, or a salt or solvate thereof.

In some embodiments, a peptide compound of formula (I) has a sequence selected among SEQ ID NO: 1 to 21 and 23 to 51, or a salt or a solvate thereof.

In some embodiments, a peptide compound of formula (I) has a sequence selected among SEQ ID NO: 1 to 10, 15, 24, 32-35, 37, 39 to 41, 43 to 49 and 51, or a salt or a solvate thereof.

In some embodiments, a peptide compound of formula (I) has a sequence selected among SEQ ID NO: 1-10, 12-18, 23-25, 28-35, 37-41 and 43-51, or a salt or a solvate thereof.

In some embodiments, a peptide compound of formula (I) has a sequence selected among SEQ ID NO: 1-10,13-17, 24-25, 28-35, 37, 39-51, or a salt or a solvate thereof.

In some embodiments, a peptide compound of formula (I) has a sequence selected among SEQ ID NO: 1-10,13-17, 24-25, 28-35, 37, 39-41, 43-51, or a salt or a solvate thereof.

In some embodiments, a peptide compound of formula (I) has a sequence selected among SEQ ID NO: 1-10, 14-16, 24, 29, 32-35, 37, 39-41, 43-49 and 51, or a salt or a solvate thereof.

In some embodiments, a peptide compound of formula (I) has a sequence selected among SEQ ID NO: 1-10, 15, 24, 32-35, 37, 39-41, 43-49 and 51, or a salt or a solvate thereof.

In some embodiments, a peptide compound of formula (I) has a sequence selected among SEQ ID NO: 1-10, 15, 24, 32-34, 37, 39-41, 43, 45-49 and 51, or a salt or a solvate thereof.

In some embodiments, a peptide compound of formula (I) has a sequence selected among SEQ ID NO: 1-8, 15, 24, 32, 34, 39, 41, 43, 45-48 and 51, or a salt or a solvate thereof.

In some embodiments, a peptide compound of formula (I) has a sequence selected among SEQ ID NO: 1-6, 15, 24, 39, 41, 43, 45-47 and 51, or a salt or a solvate thereof.

In some embodiments, a peptide compound of formula (I) has a sequence selected among SEQ ID NO: 1-2, 45 and 51, or a salt or a solvate thereof.

TABLE 2
Sequences of peptide compounds SEQ ID NOs 1 to 51

| SEQ ID | |
|---|---|
| NO. | Sequence |
| 1 | D-Lys(Ac)(@1)-Asn-Thr-Trp-Gln-Leu-Yme-Nal-Aib-Asp(@1)-Asn-Asn-NH2 |
| 2 | D-Lys(Ac)(@1)-Asn-Thr-Trp-Gln-Ala-Yme-Nal-Aib-Asp(@1)-Asn-Asn-NH2 |
| 3 | D-Lys(Ac)(@1)-Asn-Thr-Trp-Gln-Arg-Yme-Nal-Aib-Asp(@1)-Asn-Asn-NH2 |
| 4 | D-Lys(Ac)(@1)-Asn-Thr-Trp-Gln-Arg-Tyr-Nal-Aib-Asp(@1)-Asn-Asn-NH2 |
| 5 | D-Lys(Ac)(@1)-Asn-Thr-Trp-Gln-Arg-Yme-Nal-Aib-Glu(@1)-Asn-Asn-NH2 |
| 6 | Asp(Ac)(@1)-Asn-Thr-Trp-Gln-Arg-Yme-Nal-Aib-Orn(@1)-Asn-Asn-NH2 |
| 7 | D-Lys(Ac)(@1)-Asn-Thr-Trp-Gln-Leu-Yme-Nak-Aib-Asp(@1)-Asn-Asn-NH2 |
| 8 | D-Lys(Ac)(@1)-Asn-Thr-Trp-Gln-Ala-Yme-Nak-Aib-Asp(@1)-Asn-Asn-NH2 |
| 9 | Lys(Ac)(@1)-Asn-Thr-Trp-Gln-Arg-Yme-Nak-Aib-Asp(@1)-Asn-Asn-NH2 |
| 10 | D-Lys(Ac)(@1)-Asn-Thr-Wme-Gln-Arg-Yme-Nal-Lys(Ac)-Asp(@1)-Asn-Asn-NH2 |
| 11 | D-Asp(Ac)(@1)-Asn-Thr-Trp-Gln-Arg-Yme-Nak-Aib-Lys(@1)-Asn-Asn-NH2 |
| 12 | D-Asp(Ac)(@1)-Asn-Thr-Trp-Gln-Arg-Yme-Nal-Aib-Lys(@1)-Asn-Asn-NH2 |
| 13 | D-Asp(Ac)(@1)-Asn-Thr-Trp-Gln-Arg-Yme-Nal-Aib-Orn(@1)-Asn-Asn-NH2 |
| 14 | D-Lys(Ac)(@1)-Asn-Thr-Trp-Gln-Arg-Tyr-Nak-Aib-Asp(@1)-Asn-Asn-NH2 |
| 15 | D-Lys(Ac)(@1)-Asn-Thr-Trp-Gln-Arg-Yme-Nal-Aib-Asp(@1)-Asn-Asn-NH2 |
| 16 | D-Lys(Ac)(@1)-Asn-Thr-Trp-Gln-Arg-Yme-Nak-Aib-Glu(@1)-Asn-Asn-NH2 |
| 17 | D-Lys(Ac)(@1)-Asn-Thr-Trp-Gln-Arg-Yme-Nak-Lys(Ac)-Asp(@1)-Asn-Asn-NH2 |
| 18 | D-Lys(Ac)(@1)-Asn-Thr-Trp-Gln-Arg-Yme-Nak-Cit-Asp(@1)-Asn-Asn-NH2 |
| 19 | D-Lys(Ac)(@1)-Asn-Thr-Trp-Gln-Arg-Yme-Nak-Gln-Asp(@1)-Asn-Asn-NH2 |
| 20 | D-Lys(Ac)(@1)-Asn-Thr-Trp-Gln-Val-Yme-Nak-Aib-Asp(@1)-Asn-Asn-NH2 |
| 21 | D-Lys(Ac)(@1)-Asn-Thr-Trp-Gln-Aib-Yme-Nak-Aib-Asp(@1)-Asn-Asn-NH2 |
| 22 | D-orn(Ac)(@1)-Asn-Thr-Trp-Gln-Arg-Yme-Nak-Aib-Glu(@1)-Asn-Asn-NH2 |
| 23 | D-Lys(Ac)(@1)-Asn-Thr-Wme-Gln-Arg-Yme-Nak-Mle-Asp(@1)-Asn-Asn-NH2 |
| 24 | D-Lys(Ac)(@1)-Asn-Thr-Wme-Gln-Arg-Yme-Nal-Mle-Asp(@1)-Asn-Asn-NH2 |
| 25 | D-Lys(Ac)(@1)-Asn-Thr-Wme-Gln-Arg-Yme-Nal-D-tza-Asp(@1)-Asn-Asn-NH2 |
| 26 | D-Lys(Ac)(@1)-Asn-Thr-Trp-Gln-Arg-Yme-Nal-Aib-Asp(@1)-Asn-NH2 |
| 27 | D-orn(Ac)(@1)-Asn-Thr-Trp-Gln-Arg-Yme-Nal-Aib-Asp(@1)-Asn-Asn-NH2 |
| 28 | D-Lys(Ac)(@1)-Asn-Thr-Trp-Gln-Arg-Yme-Nal-Lys(Ac)-Asp(@1)-Asn-Asn-NH2 |
| 29 | D-Lys(Ac)(@1)-Asn-Thr-Trp-Gln-Arg-Yme-Nal-Cit-Asp(@1)-Asn-Asn-NH2 |
| 30 | D-Lys(Ac)(@1)-Asn-Thr-Trp-Gln-Arg-Yme-Nal-Gln-Asp(@1)-Asn-Asn-NH2 |
| 31 | D-Lys(Ac)(@1)-Asn-Thr-Trp-Gln-Val-Yme-Nal-Aib-Asp(@1)-Asn-Asn-NH2 |
| 32 | D-Lys(Ac)(@1)-Asn-Thr-Trp-Gln-Aib-Yme-Nal-Aib-Asp(@1)-Asn-Asn-NH2 |
| 33 | Asp(Ac)(@1)-Asn-Thr-Wme-Gln-Arg-Tyr-Nal-Aib-Orn(@1)-Asn-Asn-NH2 |
| 34 | Asp(Ac)(@1)-Asn-Thr-Wme-Gln-Arg-Yme-Nal-Aib-Orn(@1)-Asn-Asn-NH2 |
| 35 | Glu(Ac)(@1)-Asn-Thr-Wme-Gln-Arg-Yme-Nal-Aib-Orn(@1)-Asn-Asn-NH2 |
| 36 | Asp(Ac)(@1)-Asn-Thr-Wme-Gln-Arg-Yme-Trp-Aib-Orn(@1)-Asn-Asn-NH2 |
| 37 | Asp(Ac)(@1)-Asn-Thr-Wme-Gln-Arg-Yme-Nal-Leu-Orn(@1)-Asn-Asn-NH2 |
| 38 | Orn(Ac)(@1)-Asn-Thr-Trp-Gln-Arg-Yae-Nal-Aib-Glu(@1)-Asn-Asn-NH2 |
| 39 | D-Lys(Ac)(@1)-Asn-Thr-Trp-Gln-Agb-Yme-Nal-Aib-Asp(@1)-Asn-Asn-NH2 |
| 40 | Ahx(@1)-Asn-Thr-Trp-Gln-Agb-Yme-Nal-Aib-Glu(@1)-Asn-Asn-NH2 |
| 41 | Lys(Ac)(@1)-Asn-Thr-Trp-Gln-Arg-Yme-Nal-Thp-Asp(@1)-Asn-Asn-NH2 |
| 42 | Lys(Ac)(@1)-Asn-His-Trp-Gln-Arg-Yme-Nal-Thp-Asp(@1)-Asn-Asn-NH2 |
| 43 | Ahx(@1)-Asn-Thr-Trp-Gln-Ala-Yme-Nal-Aib-Asp(@1)-Asn-Asn-NH2 |
| 44 | Apt(@1)-Asn-Thr-Trp-Gln-Ala-Yme-Nal-Aib-Asp(@1)-Asn-Asn-NH2 |
| 45 | D-Lys(Ac)(@1)-Arg-Thr-Trp-Gln-Ala-Yme-Nal-Aib-Asp(@1)-Asn-Asn-NH2 |
| 46 | D-Lys(Ac)(@1)-Asn-Arg-Trp-Gln-Ala-Yme-Nal-Aib-Asp(@1)-Asn-Asn-NH2 |
| 47 | D-Lys(Ac)(@1)-Asn-Thr-Trp-Gln-Ala-Yme-Nal-Arg-Asp(@1)-Asn-Asn-NH2 |
| 48 | D-Lys(Ac)(@1)-Asn-Thr-Trp-Gln-Ala-Yme-Nal-Aib-Asp(@1)-Asn-Ala-NH2 |
| 49 | D-Lys(Ac)(@1)-Asn-Thr-Trp-Gln-Ala-Yme-Nal-Aib-Asp(@1)-Ala-Asn-NH2 |
| 50 | D-Lys(Ac)(@1)-Asn-Thr-Trp-Gln-Ala-Yme-Nal-Aib-Asp(@1)-Ala-Ala-NH2 |
| 51 | D-Lys(Ac)(@1)-Asn-Thr-Trp-Gln-Ala-Yme-Nal-Thp-Asp(@1)-Asn-Asn-NH2 |

(@1) indicates the formation of a lactam bridge between amino acid residues X1 and X10.
(Ac) indicates acetylation of the amino acid residue.

Peptide compounds of the disclosure display affinity for the interleukin-23 receptor and the ability to prevent or reduce the binding of IL-23 to its receptor.

The binding of peptide compounds of the disclosure to interleukin-23 prevents, reduces or inhibits the intracellular signal transduction pathway resulting in Th17 cell activation.

Peptide compounds of the disclosure are tested for interleukin-23 receptor affinity or inhibitor activity using the assays described in Methods and results shown in Table 7 herein.

In some embodiments, peptide compounds of formula (I) have a binding affinity to the interleukin 23 receptor, determined using the method described in the Examples, (i.e., Human ELISA IL-23/IL-23R) of 100 nM or less (i.e., IC₅₀<=100 nM), or of 50 nM or less (i.e., IC₅₀<=50.0 nM), or of 20 nM or less (i.e., IC₅₀<=20.0 nM), or of 10 nM or less (i.e., IC₅₀<=10.0 nM), or of 5 nM or less (i.e., IC₅₀<=5.0 nM), or of 1 nM or less (i.e., IC₅₀<=1.0 nM).

Peptide compounds of formula (I) inhibit the binding of IL-23 to IL-23 receptor with an IC₅₀ of about 50 nM or less, or of about 20 nM or less, or of about 15 nM or less, or of about 10 nM or less, or of about 5 nM or less, or of about 4 nM or less, or of about 3 nM or less, or of about 2 nM or less, or of about 1 nM or less.

Peptide compounds of formula (I) inhibit the binding of IL-23 to IL-23 receptor with an IC₅₀ ranging from about 0.1 nM to about 10 nM.

Peptide compounds of formula (I) inhibit the binding of IL-23 to IL-23 receptor with an IC₅₀ in the range of from about 0.1 nM to about 50 nM, or from about 0.2 nM to about 50 nM, or from about 0.3 nM to about 50 nM, or from about 0.5 nM to about 50 nM.

Peptide compounds of formula (I) inhibit the binding of IL-23 to the IL-23 receptor with an IC₅₀ ranging from about 0.1 nM to about 50 nM.

Peptide compounds of formula (I) inhibit the binding of IL-23 to the IL-23 receptor with an IC₅₀ ranging from about 0.1 nM to about 25 nM.

Peptide compounds of formula (I) inhibit the binding of IL-23 to the IL-23 receptor with an IC₅₀ ranging from about 0.1 nM to about 20 nM.

Peptide compounds of formula (I) inhibit the binding of IL-23 to the IL-23 receptor with an IC₅₀ ranging from about 0.1 nM to about 15 nM.

Peptide compounds of formula (I) inhibit the binding of IL-23 to the IL-23 receptor with an IC₅₀ ranging from about 0.1 nM to about 10 nM.

Peptide compounds of formula (I) inhibit the binding of IL-23 to the IL-23 receptor with an IC₅₀ ranging from about 0.1 nM to about 8 nM.

Peptide compounds of formula (I) inhibit the binding of IL-23 to IL-23 receptor with an IC₅₀ in the range of about 0.1 nM to about 6 nM.

Peptide compounds of formula (I) inhibit the binding of IL-23 to the IL-23 receptor with an IC₅₀ in the range of about 0.1 nM to about 5 nM.

Peptide compounds of formula (I) inhibit the binding of IL-23 to the IL-23 receptor with an IC₅₀ in the range of about 0.1 nM to about 4 nM.

Peptide compounds of formula (I) inhibit the binding of IL-23 to the IL-23 receptor with an IC₅₀ in the range of about 0.1 nM to about 3 nM.

Peptide compounds of formula (I) inhibit the binding of IL-23 to the IL-23 receptor with an IC₅₀ in the range of about 0.1 nM to about 2 nM.

Peptide compounds of formula (I) inhibit the binding of IL-23 to the IL-23 receptor with an IC₅₀ of about 0.1 nM to about 1 nM.

Peptide compounds of formula (I) inhibit the IL-23-induced STAT3 phosphorylation with an IC₅₀ of about 15 UM or less, or of about 10 UM or less, or of about 5 UM or less, or of about 1 UM or less.

Peptide compounds of formula (I) inhibit the IL-23-induced STAT3 phosphorylation with an IC₅₀ in the range of from about 50 nM to about 15 μM, or from about 100 nM to about 10 UM, or from about 150 nM to about 8 UM, or from about 200 nM to about 5 μM.

In a further embodiment, peptide compounds of formula (I) are sufficiently chemically stable in aqueous solution at different pH. Stability in aqueous solution is measured as a purity loss after 24 hrs at 37° C. in buffer solution.

In some embodiments, the peptide compounds of formula (I) have chemical stability in aqueous solution at pH ranging from about 1 to about 8.5, or from about 1.2 to about 7.4 as exemplified in the Examples.

A peptide compound of formula (I) is stable in aqueous solution at a pH of about 1, 1.2, 1.5, 2, 2.5, 3, 3.5, 4, 4.5, 5, 5.5, 6, 6.5, 7, 7.4, 7.5, 8, 8.5 or 9.

A peptide compound of formula (I) is stable in aqueous solution at a pH of 1.2, 6.5 or 7.4.

The stability of peptide compound of formula (I) is determined by measure of the relative purity loss as described in the Example section.

Peptide compounds of formula (I) have a stability in water at pH of about 1.2, measured as a purity loss after 24 hrs at 37° C. in buffer solution, of about 30% or less, with respect to the control, at 37° C. at 0 hour.

Peptide compounds of formula (I) have a stability in water at pH of about 1.2, measured as a purity loss after 24 hrs at 37° C., of about 25% or less, with respect to the control, at 37° C. at 0 hour.

Peptide compounds of formula (I) have a stability in water at pH of about 6.5, measured as a purity loss after 24 hrs at 37° C., of about 10% or less, or is about 5% or less, or is 2% or less, with respect to the control, at 37° C. at 0 hour.

Peptide compounds of formula (I) have a stability in water at pH of about 6.5, measured as a purity loss after 24 hrs at 37° C., of about 5% or less, with respect to the control, at 37° C. at 0 hour.

Peptide compounds of formula (I) have a stability in water at pH of about 6.5, measured as a purity loss after 24 hrs at 37° C., of about 2% or less, with respect to the control, at 37° C. at 0 hour.

In aqueous solution of pH 7.4, the relative purity loss of a peptide compound of formula (I), at 37° C. at 24 h, is about 10% or less, or is about 5% or less, or is 3% or less, with respect to the control, at 37° C. at 0 hour.

Peptide compounds of formula (I) may have a stability in water at pH of about 7.4, measured as a purity loss after 24 hrs at 37° C., of about 5% or less, with respect to the control, at 37° C. at 0 hour.

Peptide compounds of formula (I) may have a stability in water at pH of about 7.4, measured as a purity loss after 24 hrs at 37° C., of about 3% or less, with respect to the control, at 37° C. at 0 hour.

In a further embodiment, peptide compounds of formula (I) are sufficiently stable in the intestinal environment to execute a pharmacological effect.

In some embodiments, the peptide compounds of formula (I) have improved stability in gastric or intestinal environment to execute a pharmacological effect. The stability in a gastric or intestinal environment of a peptide of compound of formula (I) is determined as described in the Example section.

### Peptide Synthesis

The skilled person is aware of a variety of different methods to prepare peptide compounds. These methods include but are not limited to synthetic approaches. Thus, one way of preparing peptides is the synthesis in solution or on a solid support and subsequent isolation and purification.

A way to prepare the peptide compounds of the disclosure is solid phase synthesis on a suitable resin. Solid phase peptide synthesis is a well-established methodology (see for example: Stewart and Young, Solid Phase Peptide Synthesis, Pierce Chemical Co., Rockford, Ill., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g., a trityl resin, a chlorotrityl resin, a Wang resin or a Rink resin in which the linkage of the carboxy group (or carboxamide for Rink resin) to the resin is sensitive to acid (when Fmoc strategy is used). The polymer support must be stable under the conditions used to deprotect the α-amino group during the peptide synthesis.

After the N-terminally protected first amino acid has been coupled to the solid support, the α-amino protecting group of this amino acid is removed. The remaining protected amino acids are then coupled one after the other or with a preformed dipeptide, tripeptide or tetrapeptide in the order represented by the peptide sequence using appropriate amide coupling reagents, for example BOP, HBTU, HATU or DIC (N,N′-diisopropylcarbodiimide)/HOBt (1-hydroxybenzotriazole), wherein BOP, HBTU and HATU are used with tertiary amine bases. Alternatively, the liberated N-terminus can be functionalized with groups other than amino acids, for example carboxylic acids, etc.

Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999).

In some cases, it might be desirable to have side chain protecting groups that can selectively be removed while other side chain protecting groups remain intact. In this case the liberated functionality can be selectively functionalized. For example, a lysine may be protected with an ivDde ([1-(4,4-dimethyl-2,6-dioxocyclohex-1-ylidene)-3-methylbutyl) protecting group (S. R. Chhabra et al., Tetrahedron Lett. 39, (1998), 1603) which is labile to a very nucleophilic base, for example 4% hydrazine in DMF (dimethyl formamide). Thus, if the N-terminal amino group and all side-chain functionalities are protected with acid labile protecting groups, the ivDde group can be selectively removed using 4% hydrazine in DMF and the corresponding free amino group can then be further modified, e.g., by acylation. The lysine can alternatively be coupled to a protected amino acid and the amino group of this amino acid can then be deprotected resulting in another free amino group which can be acylated or attached to further amino acids.

Finally, the peptide is cleaved from the resin. This can be achieved by using King's cocktail (D. S. King, C. G. Fields, G. B. Fields, Int. J. Peptide Protein Res. 36, 1990, 255-266) similar cleavage cocktails known to the person skilled in the art. For example, EDT can be replaced by DODT or a mixture of TIS, water and TFA can be used. The raw material can then be purified by chromatography, e.g., preparative RP-HPLC, if necessary.

### Cyclization/Lactam Formation

Methods for lactam bond formation are known to the person skilled in the art. Exemplary methods are given in the examples.

In a further aspect the cyclization/lactam formation may be carried out after cleavage from the resin.

In a further aspect the cyclization/lactam formation may be carried out (in DMF) after cleavage from the resin and subsequent purification of the peptide by methods known to the person skilled in the art, such as preparative HPLC.

In one aspect of the cyclisation where an amide bond is formed between a primary or a secondary amine group of one amino acid and a carboxylic acid group of another amino acid, the carboxylic acid group may be activated with PyBOP or HATU in the presence of DIPEA in order to form a lactam with the unprotected amine (such as the compounds with SEQ ID NOs. 1-12, 14-21, 35, 50).

### Therapeutic Uses

A further aspect of the present disclosure relates to a peptide compound of formula (I) for use for treating and/or preventing an autoimmune or an inflammatory disease.

A peptide compound of formula (I) is for use in a subject in need thereof. Herein, the terms “subject”, “individual” and “patient” are used interchangeably. In some exemplary embodiments, the individual or subject is a human.

The present disclosure also relates to a method for treating an autoimmune or an inflammatory disease in an individual in need thereof, the method comprising at least a step of administering to said individual a peptide compound of formula (I).

The present disclosure also relates to a use of a peptide compound of formula (I) for the manufacture of a medicament for treating an autoimmune or an inflammatory disease.

The terms “disease” and “disorder” are used interchangeably and refer to any pathological or unhealthy state. In particular, a disease or disorder relates to an autoimmune or an inflammatory disease.

By “treat” or “treating” is meant to administer a compound or composition or a combination of compounds or compositions to a subject in order to eliminate a disease or disorder; arrest or slow a disease or disorder in a subject; inhibit or slow the development of a new disease or disorder in a subject; decrease the frequency or severity of symptoms and/or recurrences in a subject who currently has or who previously has had a disease or disorder; and/or prolong, i.e., increase, the lifespan of the subject. In particular, the term “treating/treatment of a disease or disorder” includes curing, shortening the duration, ameliorating, slowing down or inhibiting progression or worsening of a disease or disorder or the symptoms thereof.

By “prevent” or “preventing” is particularly meant to administer a compound or composition or a combination of compounds or compositions to a subject in order to inhibit or delay the onset of a disease or disorder in a subject.

In some embodiments, the disclosure includes methods of inhibiting IL-23 signalling by a cell expressing IL-23 receptors, comprising contacting the cells with a peptide compound of the formula (I). In some embodiments, the cell is a mammalian cell. In some embodiments, the method is performed in vitro or in vivo. In some embodiments, the inhibition of IL-23 signalling may be determined by measuring changes in phospho-STAT3 levels in the cell.

In some embodiments, the inhibition of IL-23 binding to IL-23R occurs in particular organs or tissues of the subject, e.g., the stomach, small intestine, large intestine/colon, intestinal mucosa, lamina propria, Peyer's Patches, mesenteric lymph nodes, or lymphatic ducts.

Further provided is the use of the peptide compound of formula (I), or a composition comprising the compound of formula (I), in the preparation of a pharmaceutical composition for treating an autoimmune disease or an inflammatory disease.

The autoimmune or inflammatory disease may for example be inflammatory bowel disease, such as Crohn's disease and ulcerative colitis, psoriasis, psoriatic arthritis and hidradenitis suppurativa.

In some embodiments, the disease or disorder is autoimmune inflammation and related diseases and disorders, such as multiple sclerosis, asthma, rheumatoid arthritis, inflammatory bowel diseases (IBDs), juvenile IBD, adolescent IBD, Crohn's disease, sarcoidosis, Systemic Lupus Erythematosus, ankylosing spondylitis (axial spondylarthrites), psoriatic arthritis, or psoriasis. In some embodiments, the disease or disorder is psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio or chemo-therapy, colitis associated with disorders of innate immunity as in leukocyte adhesion deficiency-1, chronic granulomatous disease, glycogen storage disease type Ib, Hermansky-Pudlak syndrome, Chediak-Higashi syndrome, Wiskott-Aldrich Syndrome, pouchitis resulting after proctocolectomy and ileoanal anastomosis, gastrointestinal cancer, pancreatitis, insulin dependent diabetes mellitus, mastitis, cholecystitis, cholangitis, primary biliary cirrhosis, viralassociated enteropathy, pericholangitis, chronic bronchitis, chronic sinusitis, asthma, uveitis, or graft versus host disease.

### Pharmaceutical Compositions

According to another aspect, pharmaceutical compositions are disclosed that include a peptide compound of formula (I), or a salt or a solvate thereof, described herein as an active ingredient. Pharmaceutical compositions of the disclosure comprise at least one peptide compound of formula (I) of the disclosure, or a pharmaceutically acceptable salt or solvate thereof, and at least one pharmaceutically acceptable excipient.

The said excipients are selected, in accordance with the pharmaceutical form and method of administration desired, from the customary excipients, which are known to a person skilled in the art.

Standard acceptable pharmaceutical carriers and their formulations are known to one skilled in the art and described, for example, in Remington: The Science and Practice of Pharmacy, (20th ed.) ed. A. R. Gennaro A. R., 2000, Lippencott Williams & Wilkins and in R. C. Rowe et al. (Ed), Handbook of Pharmaceutical excipients, PhP, May 2013 update.

In these pharmaceutical compositions for oral, sublingual, subcutaneous, intramuscular, intravenous, topical, local, intra-tracheal, intranasal, transdermal or rectal administration, the active ingredient of formula (I), or its salt, co-crystal or solvate, may be administered in a unit administration form, in a mixture with conventional pharmaceutical excipients, to human beings for the prophylaxis or treatment of the disorders or diseases or conditions that is at least partly influenced by the reduction or inhibition of the IL-23 receptor induced intracellular signaling. A desired effect may be the prevention, reduction or treatment of an inflammatory disease or an autoimmune disease, or at least one of a symptom of such diseases.

### Administration Units

The unit administration forms appropriate include oral forms such as tablets, soft or hard gel capsules, powders, granules and oral solutions or suspensions, sublingual, buccal, intratracheal, intra-ocular and intranasal administration forms, forms for inhalative, topical, transdermal, sub-cutaneous, intra-muscular or intravenous administration, rectal administration forms and implants.

When prepared in unit administration form, the pharmaceutical compositions of the disclosure typically contain from 0.01 mg to 1000 mg of peptide compound of formula (I), or a salt or a solvate thereof, described herein as the active ingredient. The amount of active ingredient that is combined with one or more excipients to produce a single unit administration form will necessarily vary depending upon the host treated and the particular route of administration. For example, a formulation intended for oral administration to humans will generally contain, for example, from 0.01 mg to 0.5 g of active ingredient compounded with an appropriate and convenient amount of excipients which may vary from about 5 to about 98 percent by weight of the total composition.

### Dosing

By the oral route the dose of a peptide compound of formula (I), or a salt or a solvate thereof, described herein as an active ingredient administered per day may reach 5 about 0.01 to 50 mg/dose, or 0.02 to 1 mg/dose or for example, from 0.0001 to 300 mg/kg body weight daily or 1 to 300 mg/kg body weight daily, taken all at once or in portions.

There may be particular cases in which higher or lower dosages are appropriate; such dosages do not depart from the scope of the disclosure. According to usual practice, the dosage that is appropriate for each patient is determined by the doctor according to the mode of administration and the weight and response of the said patient.

### EXAMPLES

The following working and prophetic examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. Thus, while the present disclosure has been described above with particularity, the following examples provide further detail in connection with what are presently deemed to be the most practical and preferred embodiments of the disclosure.

Abbreviations employed are as follows:
AA amino acid
ACN acetonitrile
Aib alpha-amino-isobutyric acid
Boc tert-butyloxycarbonyl tBu tertiary butyl
DCM dichloromethane
DIC N,N′-diisopropylcarbodiimide
DIPEA N,N-diisopropylethylamine
DMF dimethyl formamide
DODT 3,6-dioxa-1,8-octanedithiol
DPBS Dulbecco's phosphate-buffered saline
EDC 1-Ethyl-3-(3-dimethylaminopropyl) carbodiimide
EDT ethanedithiol
Fmoc fluorenylmethyloxycarbonyl g gram
HATU O-(7-azabenzotriazol-1-yl)-N,N,N,N-tetramethyluronium hexafluorophosphate
HBTU 2-(1H-benzotriazol-1-yl)-1,1,3,3-tetramethyl-uronium hexafluorophosphate
HOAt 1-hydroxy-7-azabenzotriazole
HOBt 1-hydroxybenzotriazole
HPLC High Performance Liquid Chromatography
LC/MS Liquid Chromatography/Mass Spectrometry mM millimolar
MMT monomethoxy-trityl
n.a. not available
n.d. not determined
nM nanomolar
NMP N-Methyl-2-pyrrolidone
PBS phosphate buffered saline
Pfp 2,2,4,6,7-pentamethyldihydrobenzofuran-5-sulfonyl
tBu t-butyl
TFA trifluoroacetic acid
Trt trityl/triphenymethyl
UHPLC Ultra-High Pressure Liquid Chromatography
UV ultraviolet

### Materials

Different Rink-Amide resins (e.g., 4-(2′,4′-Dimethoxyphenyl-Fmoc-aminomethyl)-phenoxyacetamido-norleucylaminomethyl resin, Merck Biosciences; 4-[(2,4-Dimethoxyphenyl) (Fmoc-amino)methyl]phenoxy acetamido methyl resin, Agilent Technologies) were used for the synthesis of peptide amides with loadings in the range of 0.2-0.7 mmol/g.

Fmoc protected natural amino acids were purchased from Protein Technologies Inc., Senn Chemicals, Merck Biosciences, Novabiochem, Iris Biotech, Bachem, Chem-Impex International or MATRIX Innovation. The following standard amino acids were used throughout the syntheses: Fmoc-L-Ala-OH, Fmoc-Arg(Pbf)-OH, Fmoc-L-Asn(Trt)-OH, Fmoc-L-Asp(OtBu)-OH, Fmoc-D-Asp(OtBu)-OH, Fmoc-L-Gln(Trt)-OH, Fmoc-L-Glu(OtBu)-OH, Fmoc-D-Glu(OtBu)-OH, Fmoc-L-Leu-OH, Fmoc-L-Lys(Boc)-OH, Fmoc-D-Lys(Boc)-OH, Fmoc-L-Phe-OH, Fmoc-L-Thr(tBu)-OH, Fmoc-L-Trp(Boc)-OH, Fmoc-L-Tyr(tBu)-OH.

In addition, the following special amino acids were purchased from the same suppliers as above: Fmoc-Aib-OH, Fmoc-L-1-Nal-OH, Fmoc-L-2-Nal-OH, Fmoc-L-Phe(2-Ae-Boc)-OH, Fmoc-L-Phe(4-Ome)-OH, Fmoc-L-Orn(Boc)-OH, Fmoc-D-Orn(Boc)-OH, Fmoc-L-Trp(Me)-OH, Fmoc-L-alpha-Me-Leu-OH, Fmoc-beta(4-thiazolyl)-Ala-OH, Fmoc-4-amino-tetrahydropyran-4-carboxylic acid, Fmoc-Cit-OH, Fmoc-Agb(Pbf)-OH, Fmoc-Ahx-OH and Fmoc-Apt-OH.

### Example 1: General Synthesis of Peptide Compounds

The solid phase peptide syntheses were performed on a Prelude Peptide Synthesizer (Protein Technologies) or a similar automated synthesizer using standard Fmoc chemistry and HBTU/DIPEA or HATU/DIPEA activation. DMF was used as the solvent.
Deprotection: 20% piperidine/DMF for 2×2.5 min.
Washes: 7×DMF.
Coupling: May 5, 2013 200 mM AA/500 mM HBTU in DMF/2M DIPEA in NMP 2× for 20 min. Washes: 5×DMF.

HBTU/DIPEA activation was used for all standard couplings.

For N-terminal acetylated peptides, the N-terminal Fmoc protecting group was removed and the peptide was treated twice with 10% solution of acetic acid anhydride and DIPEA for 20 minutes with shaking.

The peptides were cleaved from the resin with King's cleavage cocktail consisting of 82.5% TFA, 5% phenol, 5% water, 5% thioanisole, and 2.5% EDT or a modified cleavage cocktail consisting of 82.5% TFA, 5% phenol, 5% water, 5% thioanisole, and 2.5% DODT. The crude peptides were then precipitated in diethyl or diisopropyl ether, centrifuged, and lyophilized. Peptides were analyzed by analytical HPLC and checked by ESI mass spectrometry. Crude peptides were purified by a conventional preparative RP-HPLC purification procedure.

### Synthesis of the Peptide IL-23 Receptor Inhibitor SEQ. ID NO. 24

D-Lys(Ac)(@1)-Asn-Thr-Wme-Gln-Arg-Yme-Nal-Mle-Asp(@1)-Asn-Asn-NH2

FMOC solid phase peptide synthesis techniques were used with Rink Amide AM Resin LL and a Prelude synthesizer with standard Fmoc protection synthesis conditions.

Swelling of Resin: 0.1 mmol of Rink Amide AM Resin LL (0.345 g, 0.29 mmol/g loading) was transferred to a 25 ml peptide vessel with filter frit. The resin was washed 3× with 4 ml DMF for 5 min.

### Step I: Coupling of Fmoc-Asn(Trt)-OH (N):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Asn(Trt)-OH (5 eq, 2.55 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with nitrogen prior to starting the next deprotection-coupling cycle.

### Step II: Coupling of Fmoc-Asn(Trt)-OH (N):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Asn(Trt)-OH (5 eq, 2.55 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step III: Coupling of Fmoc-Asp(OtBu)-OH (D):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Asp(OtBu)-OH (5 eq, 2.5 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step IV: Coupling of Fmoc-L(αMe)-OH (Mle):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-L(αMe)-OH (5 eq, 2.5 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step V: Coupling of Fmoc-(2S)-2-Amino-3-(2-Naphthyl)Propanoic Acid (Nal):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-(2S)-2-Amino-3-(2-naphthyl) propanoic acid (2.5 eq, 2.5 ml, 100 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step VI: Coupling of Fmoc-F (4-OMe)-OH (Yme):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-F (4-OMe)-OH (2.5 eq, 2.5 ml, 100 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step VII: Coupling of Fmoc-Arg(Pbf)-OH Acid (Arg):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Arg(Pbf)-OH (2.5 eq, 2.5 ml, 100 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step VIII: Coupling of Fmoc-Gln(Trt)-OH (Q):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Gln(Trt)-OH (5 eq, 2.55 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step IX: Coupling of Fmoc-Trp(Me)-OH (Wme):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Trp(Me)-OH (5 eq, 2.5 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step X: Coupling of Fmoc-Thr(tBu)-OH (T):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Thr(tBu)-OH (2.5 eq, 2.5 ml, 100 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

### Step XI: Coupling of Fmoc-Asn(Trt)-OH (N):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of Fmoc-Asn(Trt)-OH (5 eq, 2.55 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ prior to starting the next deprotection-coupling cycle.

Coupling of εFmoc-D-Lys(αAc)—OH (k(Ac)):

For the deprotection of the resin bound Fmoc group 4 ml of 20% piperidine in DMF was added to the swollen resin and was mixed for 2.5 min by bubbling with nitrogen prior to draining and this process was repeated once.

After deprotection the resin was washed 7× with 4 ml DMF with mixing. A solution of &Fmoc-D-Lys(αAc)—OH (5 eq, 2.55 ml, 200 mM in DMF) was added to the resin followed by HATU (5 eq, 1 ml, 500 mM in DMF) and DiPEA (13 eq, 0.65 ml, 2 M in NMP). The mixture was mixed by bubbling with nitrogen for 40 min prior draining. The resin was washed once with 4 ml DMF before the coupling process was repeated once. The resin was washed 5× with 4 ml DMF by bubbling with N₂ and deprotection of the resin bound Fmoc group was realized by adding 4 ml of 20% piperidine in DMF to the swollen resin and mixing for 2.5 min by bubbling with N₂ prior to draining and repeated this process once.

The constructed peptide was isolated from the resin and protecting groups by cleavage with Kings cocktail followed by precipitation with diethyl ether.

The crude peptide was dissolved in DMF (10 mg/ml) followed by the addition of PyBOP (1.2 eq) and DIPEA (4 eq). The mixture was stirred at room temperature for 2 hours until the lactamization was complete. This was followed by purification by RP-HPLC. Lyophilization of pure fractions gives the desired peptide IL-23 receptor inhibitor SEQ. ID NO. 24.

### Example 2: Peptide Cyclisation

Alternatively, the peptide was dissolved in DMF, cooled to 0° C. before 20 eq. NaHCO₃, 3 eq. HOAT and 2.5 eq EDC.HCl was added. The reaction was stirred and allowed to slowly equilibrate to room temperature overnight. NaHCO₃ was filtered over gravity and the soluble fraction dried under rotary evaporation. The crude cyclic peptide was dissolved in ACN:Water and freeze-dried. The desired cyclic peptide product was isolated by a conventional preparative RP-HPLC purification procedure.

By this method peptide compounds obtained are shown in Table 3:

TABLE 3
Synthesized peptide compounds

| Seq ID | |
|---|---|
| NO | Formula |
| 1 | 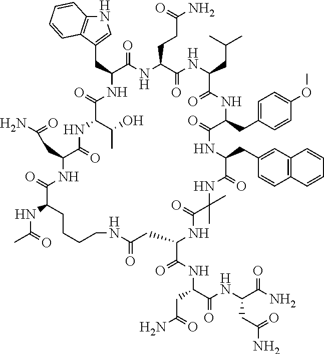 [SMILES: COc1ccc(C[C@@H]2NC(=O)[C@H](CC(C)C)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3ccc4ccccc4c3)NC2=O)cc1] |
| 2 | 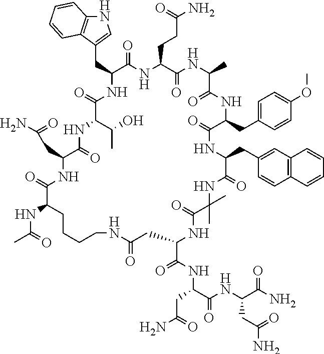 [SMILES: COc1ccc(C[C@@H]2NC(=O)[C@H](C)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3ccc4ccccc4c3)NC2=O)cc1] |
| 3 | 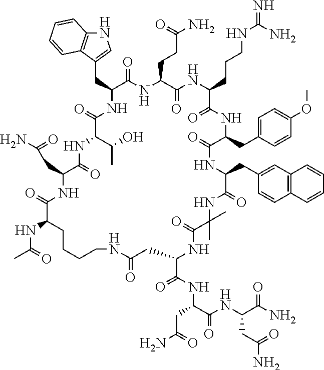 [SMILES: COc1ccc(C[C@@H]2NC(=O)[C@H](CCCNC(=N)N)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3ccc4ccccc4c3)NC2=O)cc1] |
| 4 | 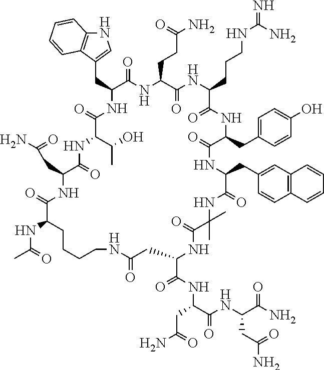 [SMILES: CC(=O)N[C@@H]1CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc2ccc3ccccc3c2)NC(=O)[C@H](Cc2ccc(O)cc2)NC(=O)[C@H](CCCNC(=N)N)NC(=O)[C@H](CCC(N)=O)NC(=O)[C@H](Cc2c[nH]c3ccccc23)NC(=O)[C@H]([C@@H](C)O)NC(=O)[C@H](CC(N)=O)NC1=O] |
| 5 | 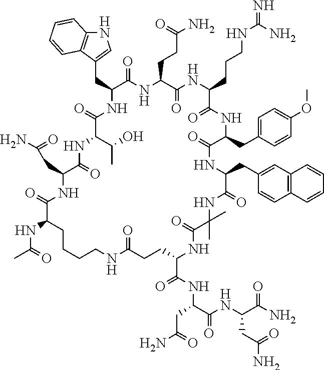 [SMILES: COc1ccc(C[C@@H]2NC(=O)[C@H](CCCNC(=N)N)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)CC[C@@H](C(=O)N[C@@H](CC(N)=O)C(=O)N[C@@H](CC(N)=O)C(N)=O)NC(=O)C(C)(C)NC(=O)[C@H](Cc3ccc4ccccc4c3)NC2=O)cc1] |
| 6 | 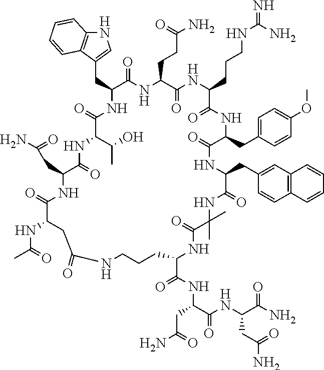 [SMILES: COc1ccc(C[C@@H]2NC(=O)[C@H](CCCNC(=N)N)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@@H](NC(C)=O)CC(=O)NCCC[C@@H](C(=O)N[C@@H](CC(N)=O)C(=O)N[C@@H](CC(N)=O)C(N)=O)NC(=O)C(C)(C)NC(=O)[C@H](Cc3ccc4ccccc4c3)NC2=O)cc1] |
| 7 | 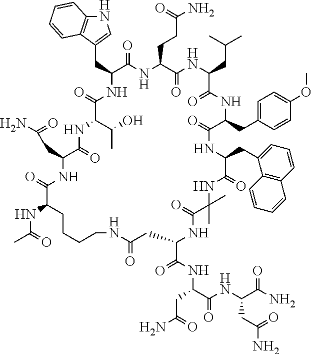 [SMILES: COc1ccc(C[C@@H]2NC(=O)[C@H](CC(C)C)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3cccc4ccccc34)NC2=O)cc1] |
| 8 | 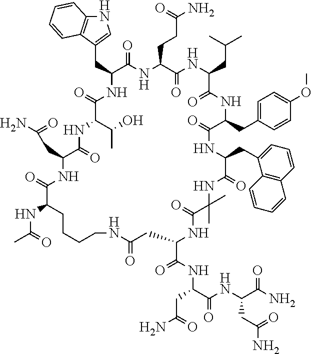 [SMILES: COc1ccc(C[C@@H]2NC(=O)[C@H](CC(C)C)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3cccc4ccccc34)NC2=O)cc1] |
| 9 | 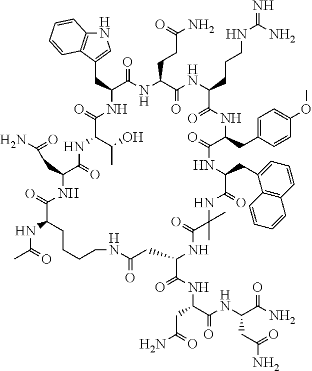 [SMILES: COc1ccc(C[C@@H]2NC(=O)[C@H](CCCNC(=N)N)NC(=O)[C@H](CCC(N)=O)NC(=O)[C@H](Cc3c[nH]c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)C(C)(C)NC(=O)[C@H](Cc3cccc4ccccc34)NC2=O)cc1] |
| 10 | 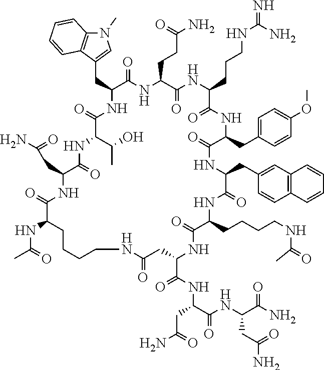 [SMILES: COc1ccc(C[C@@H]2NC(=O)[C@H](CCCNC(=N)N)NC(=O)[C@H](CCC(N)=O)NC(=O)[C@H](Cc3cn(C)c4ccccc34)NC(=O)[C@H]([C@@H](C)O)NC(=O)[C@H](CC(N)=O)NC(=O)[C@H](NC(C)=O)CCCCNC(=O)C[C@@H](C(=O)N[C@@H](CC(N)=O)C(=O)N[C@@H](CC(N)=O)C(N)=O)NC(=O)[C@H](CCCCNC(C)=O)NC(=O)[C@H](Cc3ccc4ccccc4c3)NC2=O)cc1] |
| 11 to 51 | See Table 2 |

### Example 3: Analytical HPLC/UHPLC

### Method A: Detection at 214 nm

HPLC: Waters i-class UPLC
column: Waters ACQUITY UPLC® CSH™ C18 1.7 μm (75×2.1 mm) at 50° C. solvent: H2O+0.05% TFA: ACN+0.045% TFA (flow 0.7 ml/min)
gradient: 95:5 (0 min) to 95:5 (1 min) to 20:80 (12 min) to 5:95 (12.5 min) to 5:95 (13.5 min) to 95:5 (14 min) to 95:5 (16 min)
with mass analyser: Waters Xevo G2-XS QTof, electrospray positive ion mode, mass range: 300-3200 m/z, resolution mode

### Method B: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 80:20 (0 min) to 80:20 (3 min) to 25:75 (23 min) to 2:98 (23.5 min) to 2:98 (30.5 min) to 80:20 (31 min) to 80:20 (37 min)
with mass analyser: Agilent 6230 Accurate-Mass TOF, Agilent Dual Jet Stream ESI+, mass range: 300-3200 m/z, extended dynamic range mode (2 Ghz)

### Method C: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 95:5 (0 min) to 95:5 (3 min) to 40:60 (23 min) to 2:98 (23.5 min) to 2:98 (30.5 min) to 95:5 (31 min) to 95:5 (37 min)
with mass analyser: Agilent 6230 Accurate-Mass TOF, Agilent Dual Jet Stream ESI, mass range: 300-3200 m/z, extended dynamic range mode (2 Ghz)

### Method D: Detection at 214 nm

HPLC: Agilent 1290 Infinity II UPLC
column: Waters ACQUITY UPLC® CSH™ C18 1.7 μm (150×2.1 mm) at 50° C. solvent: H2O+0.05% TFA: ACN+0.035% TFA (flow 0.5 ml/min)
gradient: 99:1 (0 min) to 99:1 (3 min) to 70:30 (18 min) to 2:98 (18.5 min) to 2:98 (25.5 min) to 99:1 (26 min) to 99:1 (32 min)
with mass analyser: Agilent 6230 Accurate-Mass TOF, Agilent Dual Jet Stream ESI, mass range: 300-3200 m/z, extended dynamic range mode (2 Ghz)

TABLE 4
List of synthesized peptides and comparison of
calculated vs. found molecular weight (MIM =
monoisotropic mass [Da]) and used HPLC/UHPLC method

| Seq ID | Calc. mass | Found mass | Retention time | |
|---|---|---|---|---|
| No | MIM | MIM | [min] | Method |
| 1 | 1613.763 | 1613.71 | 12.880 | B |
| 2 | 1571.716 | 1571.72 | 11.560 | B |
| 3 | 1656.780 | 1656.79 | 15.520 | C |
| 4 | 1642.764 | 1642.77 | 14.140 | C |
| 5 | 1670.795 | 1670.80 | 15.230 | C |
| 6 | 1642.764 | 1642.77 | 14.740 | C |
| 7 | 1613.763 | 1613.761 | 11.269 | B |
| 8 | 1571.716 | 1571.71 | 16.700 | B |
| 9 | 1656.780 | 1656.79 | 15.570 | C |
| 10 | 1755.848 | 1755.854 | 9.212 | B |
| 11 | 1670.795 | 1670.80 | 15.410 | C |
| 12 | 1656.780 | 1656.79 | 15.320 | C |
| 13 | 1642.764 | 1642.77 | 15.160 | C |
| 14 | 1642.764 | 1642.766 | 7.406 | B |
| 15 | 1656.780 | 1656.79 | 15.470 | C |
| 16 | 1670.795 | 1670.798 | 8.317 | B |
| 17 | 1741.832 | 1741.834 | 8.509 | B |
| 18 | 1728.812 | 1728.82 | 14.880 | C |
| 19 | 1699.786 | 1699.79 | 14.880 | C |
| 20 | 1599.747 | 1599.747 | 11.114 | B |
| 21 | 1585.731 | 1585.74 | 17.230 | C |
| 22 | 1656.780 | 1656.783 | 8.403 | B |
| 23 | 1712.842 | 1712.85 | 17.200 | C |
| 24 | 1712.842 | 1712.848 | 10.541 | B |
| 25 | 1739.763 | 1739.77 | 16.680 | C |
| 26 | 1542.737 | 1542.75 | 10.580 | B |
| 27 | 1642.764 | 1642.77 | 15.650 | C |
| 28 | 1741.832 | 1741.84 | 15.200 | C |
| 29 | 1728.812 | 1728.82 | 14.900 | B |
| 30 | 1699.786 | 1699.79 | 14.960 | C |
| 31 | 1599.747 | 1599.75 | 17.990 | C |
| 32 | 1585.731 | 1585.755 | 10.732 | B |
| 33 | 1642.764 | 1642.77 | 9.060 | B |
| 34 | 1656.780 | 1656.79 | 10.140 | B |
| 35 | 1670.795 | 1670.81 | 10.630 | B |
| 36 | 1645.775 | 1645.79 | 8.940 | B |
| 37 | 1684.811 | 1684.82 | 11.060 | B |
| 38 | 1685.806 | 1685.814 | 15.970 | D |
| 39 | 1642.764 | 1642.77 | 9.910 | B |
| 40 | 1599.758 | 1599.76 | 9.660 | B |
| 41 | 1698.790 | 1698.80 | 15.620 | C |
| 42 | 1734.802 | 1734.81 | 8.040 | B |
| 43 | 1514.694 | 1514.70 | 11.190 | B |
| 44 | 1500.679 | 1500.68 | 11.250 | C |
| 45 | 1613.774 | 1613.77 | 9.970 | B |
| 46 | 1626.769 | 1626.78 | 9.490 | B |
| 47 | 1642.764 | 1642.77 | 9.580 | B |
| 48 | 1528.710 | 1528.72 | 12.230 | B |
| 49 | 1528.710 | 1528.72 | 12.350 | B |
| 50 | 1485.704 | 1485.72 | 13.700 | B |
| 51 | 1613.726 | 1613.69 | 11.440 | B |

### Example 4: General Preparative HPLC Purification Procedure

The crude peptides were purified either on an Äkta Purifier System, a Jasco semiprep HPLC System, Waters Autopurification System, an Agilent 1100 HPLC system or a similar HPLC system. Preparative RP-C18-HPLC columns of different sizes and with different flow rates were used depending on the amount of crude peptide to be purified, e.g., the following columns have been used: Waters Xselect CSH C18 OBD Prep 5 μm 30×250 mm, Waters SunFire C18 OBD Prep 5 μm 30×250 mm, Waters SunFire C18 OBD Prep 5 μm 50×150 mm, and Phenomenex Luna Prep C18 5 μm 21.2×250 mm. Acetonitrile (B) and water+0.1% TFA (A) or water+0.1% FA (A) were employed as eluents. Product-containing fractions were collected and lyophilized to obtain the purified product, typically as TFA salt.

Alternatively, the peptides can be isolated as acetate salts via the following procedure: The peptide was dissolved in water and the solution adjusted to pH 7.05 with NaHCO3. Then, the dissolved compound was purified with a RP Kinetex 21, 2×250 mm (Column Volume CV 88 ml, 5 μm, C18, 100 A, Äkta avant 25): The column was equilibrated with solvent A (3×CV), the compound was injected and then washed with a mixture of solvent A (95%) and solvent B (5%) with 3 CV. Then, a gradient solvent A:B (95:5) to A:B (20:80) was run with 15 CV. The purified peptide was collected and lyophilized.

### Example 5: Chemical Stability Assessment of Example Compounds

Purity of a compound batch was determined through UHPLC/MS prior to chemical stability measurement using Method A.

For stability assessment, the target concentration was 300 μM pure compound. Therefore, solutions from solid samples were prepared in the following buffer systems with a concentration of ~300 UM compound based on the previously determined % purity. The solid samples were obtained by drying down aliquots of a freshly prepared acetonitrile stock solution using a THERMO SPEEDVAC vacuum centrifuge. The dry down time was 3 hours.

The following buffer systems were applied:
Solubility buffer system A) 100 mM phosphate buffer pH 7.4.
Solubility buffer system B) 100 mM phosphate buffer pH 6.5.
Solubility buffer system C) 100 mM HCl pH 1.2.

Prepared solutions were filtered through 0.22 M pore size and filled into sterilized glass containers under laminar flow conditions.

Glass containers were stored for 28 days at 5 and 40° C. After this time course, the samples were centrifuged for 15 min at 2500 RCF. Then 1.5 μl of the undiluted supernatant were analysed with UHPLC-UV.

The chemical stability was rated through the relative loss of purity calculated by the equation:

[(purity after 0 hours at 37° C.)(purity after 24 hours at 37° C.)]/(purity after 0 hours at 37° C.)]*100%.

The purity is calculated as [(peak area peptide)/(total peak area)]*100%.

Results are shown in Table 5.

### Tables 5A, 5B and 5C: Chemical Stability (Relative Purity Loss) of Peptide Compounds in Buffer Formulations

TABLE 5A
pH 1.2

| | | | | Relative | Mass |
|---|---|---|---|---|---|
| | | Conc. | Conc. | Purity | Balance |
| SEQ | Purity | [mg/ml] | [mg/ml] | Loss [%] | [%] |
| ID NO | [%] | 0 h | 24 h 37° C. | 24 h 37° C. | 24 h 37° C. |
| 3 | 98.4 | 1.5 | 1.1 | 23.2 | −3.4 |
| 4 | 98.3 | 1.4 | 1.0 | 22.9 | −2.3 |
| 6 | 97.6 | 1.6 | 1.1 | 27.3 | −3.8 |
| 7 | 93.1 | 1.1 | 0.7 | 26.5 | −9.6 |
| 8 | 89.2 | 0.2 | 0.2 | 26.2 | −2.5 |
| 9 | 97.1 | 1.5 | 1.1 | 22.3 | −1.9 |

TABLE 5B
pH 6.5

| | | | | Relative | Mass |
|---|---|---|---|---|---|
| | | Conc. | Conc. | Purity | Balance |
| SEQ | Purity | [mg/ml] | [mg/ml] | Loss [%] | [%] |
| ID NO | [%] | 0 h | 24 h 37° C. | 24 h 37° C. | 24 h 37° C. |
| 3 | 98.6 | 1.2 | 1.1 | 1.1 | −3.6 |
| 4 | 100 | 1.4 | 1.3 | 0.7 | −4.6 |
| 6 | 99.5 | 1.4 | 1.3 | 0.6 | −1.4 |
| 7 | 95.1 | 1.5 | 1.5 | 0.3 | −3.9 |
| 8 | 87.6 | 0.2 | 0.2 | −1.4 | 4.1 |
| 9 | 98.9 | 1.2 | 1.2 | 1.2 | −1.8 |

TABLE 5C
pH 7.4

| | | | | Relative | Mass |
|---|---|---|---|---|---|
| | | Conc. | Conc. | Purity | Balance |
| SEQ | Purity | [mg/ml] | [mg/ml] | Loss [%] | [%] |
| ID NO | [%] | 0 h | 24 h 37° C. | 24 h 37° C. | 24 h 37° C. |
| 3 | 98.2 | 1.4 | 1.3 | 3.6 | −3.1 |
| 4 | 99.8 | 1.4 | 1.3 | 2.7 | −2.2 |
| 6 | 99.2 | 1.4 | 1.3 | 1.4 | −4.8 |
| 7 | 95.5 | 1.1 | 1.0 | 4.7 | −2.3 |
| 8 | 86.7 | 0.2 | 0.2 | 2.0 | 4.6 |
| 9 | 98.5 | 1.5 | 1.4 | 2.6 | −3.2 |

Buffer composition: 100 mM HCl pH 1.2, 100 mM phosphate buffer pH 6.5, 100 mM phosphate buffer pH 7.4

### Example 6: Protease Stability Assessment of Peptides

Studies were carried out in simulated intestinal fluid (SIF) and simulated gastric fluid (SGF) to evaluate gastric stability of the peptide compounds of the disclosure.

For clarification, the term “Pepsin-SGF” refers to a solution of simulated gastric fluid containing pepsin whereas “SGF media” refers to a solution of simulated gastric fluid without pepsin. Pepsin-SGF was prepared by dissolving 320 mg Pepsin (Merck Millipore) in 100 mL SGF media concentrate (ProSense).

Similarly, the term “Pancreatin-SIF” refers to a solution of simulated intestinal fluid containing pancreatin whereas “SIF-media” refers to a solution of simulated intestinal fluid without pancreatin. Pancreatin-SIF was prepared by dissolving 1 mg Pancreatin (MP Biomedicals) in 10 mL SIF media concentrate (ProSense).

Peptide compounds were first dissolved at 1 mM stock concentration in 0.01 HCl (+/10% of DMSO) and were than diluted further with water to a stock concentration of 20 M. Final compound concentration in the incubation was 1 μM.

To 76 μL of the Pepsin-SGF buffer, 4 L of the 20 μM peptide stock solution were added and incubated at 37° C. At each timepoint (0, 60, 120 and 240 minutes) the reaction was quenched with addition of 80 μL Ethanol (0.1% HCOOH). The samples were centrifuged for 20 minutes and an aliquot of the supernatant 100 μL were transferred to a LoBind-MTP plate and analyzed by LCMS/MS. The peak area response was calculated at each time point for each test compound. As internal reference, the peak area response of the same test compound at TO was calculated. The percent remaining at each timepoint was calculated based on the peak area response ratio of the test compound at TO. Time 0 was set to 100%, and all later timepoints were calculated relative to time 0.

Peptide compounds were first dissolved at 1 mM stock concentration in 0.01 HCl (+/10% of DMSO) and were than diluted further with water to a stock concentration of 20 μM. Final compound concentration in the incubation was 1 μM.

To 76 μL of the Pepsin-SGF buffer, 4 μL of the 20 UM compound stock solution were added and incubated at 37° C. At each timepoint (0, 60, 120 and 240 minutes) the reaction was quenched with addition of 80 μL Ethanol (0.1% HCOOH). The samples were centrifuged for 20 minutes and an aliquot of the supernatant 100 μL were transferred to a LoBind-MTP plate and analyzed by LCMS/MS. The peak area response was calculated at each time point for each test compound. As internal reference, the peak area response of the same test compound at TO was calculated. The percent remaining at each timepoint was calculated based on the peak area response ratio of the test compound at TO. Time 0 was set to 100%, and all later timepoints were calculated relative to time 0.

TABLE 6
Protease stability in simulated intestinal fluids (FaSSIF)
expressed as remaining percentage of peptide compound.

| SEQ | FaSSIF | FaSSIF | FaSSIF | FaSSIF |
|---|---|---|---|---|
| ID NO | 0 (min) | 60 (min) | 120 (min) | 240 (min) |
| 11 | 100 | 4 | 7 | 2 |
| 13 | 100 | 2 | 0 | 3 |
| 18 | 100 | 8 | n.a. | 11 |
| 21 | 100 | 96 | 98 | 95 |

### Example 7: Assay for Specific Human IL-23R Inhibitors

Antagonism of peptide compounds for human interleukin-23 receptor inhibitors, was determined by an ELISA assay.

A 384-well plate was coated with 50 μl/well of human IL-23 at a final concentration of 1 μg/ml and incubated overnight at 4° C. The wells were washed 3 times with 80 μl wash buffer and blocked with 80 μl blocking buffer for 60 minutes at room temperature and washed again. 20 μl of serially diluted test peptides was added to each well and subsequently 20 μl of recombinant human IL-23R-Fc chimera at final concentration of 0.1 μg/ml. The plate was incubated for 60 minutes at room temperature. After the wells were washed, bound IL-23R-Fc was detected with goat anti-hu-lgG1-HRP antibody. Signals were visualized with QuantaBlu Fluorogenic Peroxidase Substrate.

For selectivity of the IL-23R inhibitor compounds binding to human IL12Rβ1, recombinant human IL-12Rβ1-Fc was used for the ELISA assay.

For selectivity of the IL-23R inhibitor compounds binding to mouse IL-23R, recombinant mouse IL-23R-Fc chimera was used and bound mouse IL-23R-FC was detected with goat anti-mouse-lgG1-HRP antibody.

TABLE 7
IC₅₀ values of IL-23 binding to IL-23R with
peptide compounds as measured by ELISA

| | Human ELISA |
|---|---|
| | IL-23/IL-23R binding inhibition |
| SEQ ID NO | IC₅₀ [nM] |
| 1 | 0.63 |
| 2 | 0.69 |
| 3 | 1.55 |
| 4 | 1.59 |
| 5 | 1.95 |
| 6 | 1.96 |
| 7 | 2.47 |
| 8 | 2.74 |
| 9 | 3.55 |
| 10 | 3.60 |
| 11 | 18.92 |
| 12 | 14.87 |
| 13 | 6.24 |
| 14 | 5.42 |
| 15 | 1.04 |
| 16 | 5.74 |
| 17 | 7.87 |
| 18 | 11.75 |
| 19 | 15.62 |
| 20 | 15.90 |
| 21 | 16.90 |
| 22 | 48.40 |
| 23 | 12.01 |
| 24 | 1.21 |
| 25 | 9.61 |
| 26 | 18.83 |
| 27 | 19.73 |
| 28 | 9.03 |
| 29 | 5.63 |
| 30 | 9.64 |
| 31 | 8.27 |
| 32 | 2.68 |
| 33 | 3.36 |
| 34 | 2.16 |
| 35 | 4.55 |
| 36 | 19.47 |
| 37 | 3.57 |
| 38 | 12.50 |
| 39 | 1.46 |
| 40 | 3.11 |
| 41 | 1.47 |
| 42 | 18.10 |
| 43 | 1.44 |
| 44 | 4.66 |
| 45 | 0.96 |
| 46 | 1.38 |
| 47 | 1.34 |
| 48 | 2.06 |
| 49 | 3.13 |
| 50 | 8.29 |
| 51 | 0.78 |

### Example 8: Inhibition of IL-23 Induced STAT3 Signaling in HEKBlue™ IL-23 Cells

HEK-Blue™ IL-23 cells (Invivogen) are designed for the detection of bioactive human (hIL-23) and murine IL-23 (mIL-23) by monitoring the activation of the STAT3 pathway. Binding of IL-23 to its receptor on the surface of HEK-Blue™ IL-23 cells triggers a signaling cascade leading to the activation of STAT3 and the subsequent production of secreted embryonic alkaline phosphatase (SEAP). This can be readily assessed using QUANTI-Blue™ Solution, a SEAP detection reagent.

HEK-Blue™ IL-23 cells were cultured as described by Invivogen.

On the first day of assay 10 μL of the HEK-Blue IL-23 cell suspension (final concentration: 10,000 cells/well) and 10 μl of serially diluted compound solutions were dispensed into a 384-well assay plate. After pre-incubation of the reaction mixtures at 37° C./5% CO2/95% humidity for 60 minutes, 10 μl of the humanIL-23 cytokine (final concentration: 0.25 ng/ml) was added to each well for the STAT3 signaling activation. The plate was incubated overnight at 37° C./5% CO2/95% humidity.

On the second day 20 μl per well of Quanti-Blue detection solution (prepared according to the manufacture instruction) was dispensed into a new 384-well plate and followed by the transfer of 5 μl supernatant of the overnight incubated cell/sample mixtures. After incubation of 45 min at room temperature signals were measured with PHERAstar (BMG) at OD 620-655 nm.

Dose response data were fitted with XLfit.

TABLE 8
IC₅₀ values of IL-23 induced pSTAT3 as measured
by SEAP reporter of phosphorylation of STAT3

| | Inhibition of IL-23 induced pSTAT3 |
|---|---|
| SEQ ID NO | (IC₅₀ nM) |
| 1 | 333.77 |
| 2 | 233.69 |
| 3 | 430.36 |
| 4 | 339.00 |
| 5 | 432.38 |
| 6 | 366.00 |
| 7 | 850.00 |
| 8 | 943.00 |
| 9 | 1610.00 |
| 10 | 1442.84 |
| 11 | 9280.00 |
| 12 | 6590.00 |
| 13 | 1150.00 |
| 14 | 1380.00 |
| 15 | 426.00 |
| 16 | 8010.00 |
| 17 | 5650.00 |
| 18 | 10000.00 |
| 19 | 9600.00 |
| 20 | 10000.00 |
| 21 | 7500.00 |
| 23 | 1960.00 |
| 24 | 385.03 |
| 25 | 3830.00 |
| 26 | 8113.08 |
| 28 | 2208.31 |
| 29 | 2722.81 |
| 30 | 6054.06 |
| 31 | 128.00 |
| 32 | 320.00 |
| 38 | 1572.64 |
| 41 | 220.53 |

### Example 9: Solubility Testing of Example at Different pH

Prior to the solubility measurement of a compound batch, its purity was determined through UHPLC/MS Method A.

For solubility testing the target concentration was 1 mg pure compound/ml. Therefore, solutions from solid samples were prepared in a buffer system at a concentration of 1 mg/mL compound. The solid samples were obtained by drying down aliquots of a dimethyl sulfoxide stock solution using a Eppendorf Speedvac vacuum centrifuge. The dry down time was 3 hours.
Solubility buffer system A) 50 mM phosphate buffer pH 7.4.
Solubility buffer system B) 50 mM phosphate buffer pH 6.5.
Solubility buffer system C) 50 mM HCl pH 1.2.

UHPLC-UV was performed after 2 hours of gentle agitation from the supernatant, which was obtained after 10 min of centrifugation at 1500 RCF (relative centrifugal acceleration).

The solubility was determined by the comparison of the UV peak area of 2 μL-injections of a buffered sample diluted 1:5 with a standard curve of a dimethyl sulfoxide reference sample with known concentration. The different extinction coefficients were taken into account for the calculation.

### REFERENCES

Atherton E. and R. C. Sheppard, Solid Phase Peptide Synthesis. A Practical Approach, Oxford-IRL Press, New York, 1989
Bis R L, Mallela K M. Antimicrobial preservatives induce aggregation of interferon alpha-2a: the order in which preservatives induce protein aggregation is independent of the protein. Int J Pharm. 2014 Sep. 10; 472(1-2):356-61. doi: 10.1016/j.ijpharm.2014.06.044. Epub 2014 Jun. 27. PMID: 24974985; PMCID: PMC4268133.
Brayden D J, Mrsny R J. Oral peptide delivery: prioritizing the leading technologies. Ther Deliv. 2011 December; 2(12):1567-73. doi: 10.4155/tde.11.114. PMID: 22833982.
David B. Troy, Paul Beringer. Remington: The Science and Practice of Pharmacy Lippincott Williams & Wilkins, 2006 2393 pages
Kamerzell T J, Esfandiary R, Joshi S B, Middaugh C R, Volkin D B. Protein-excipient interactions: mechanisms and biophysical characterization applied to protein formulation development. Adv Drug Deliv Rev. 2011 October; 63(13):1118-59. doi: 10.1016/j.addr.2011.07.006. Epub 2011 Jul. 29. PMID: 21855584.
King D S, Fields C G, Fields G B. A cleavage method which minimizes side reactions following Fmoc solid phase peptide synthesis. Int J Pept Protein Res. 1990 September; 36(3):255-66. doi: 10.1111/j.1399-3011.1990.tb00976.x. PMID: 2279849.
Rowe, Raymond C. et al. “Handbook of Pharmaceutical Excipients.” (1994).
Siri Ram Chhabra, Bhupinder Hothi, David J. Evans, Peter D. White, Barrie W. Bycroft, Weng C. Chan, An appraisal of new variants of Dde amine protecting group for solid phase peptide synthesis, Tetrahedron Letters, Volume 39, Issue 12, 1998, Pages 1603-1606, ISSN 0040-4039, https://doi.org/10.1016/S0040-4039(97) 10828-0.
Sayago C, Gonzalez Valcarcel I C, Qian Y, Lee J, Alsina-Fernandez J, Fite N C, Carrillo J J, Zhang F F, Chalmers M J, Dodge J A, Broughton H, Espada A. Deciphering Binding Interactions of IL-23R with HDX-MS: Mapping Protein and Macrocyclic Dodecapeptide Ligands. ACS Chem 2018 Med Lett. August 1; 9(9):912-916. doi: 10.1021/acsmedchemlett.8b00255. PMID: 30258540; PMCID: PMC6142055.
Stewart John Morrow and Young Janis Dillaha. Solid phase peptide synthesis. Pierce Chemical Co., Rockford, Ill. et ©1984
Theodora W. Greene Ph.D., Peter G. M. Wuts Ph.D., Protective Groups in Organic Synthesis, Third Edition. Copyright © 1999 by John Wiley & Sons, Inc. Print ISBN: 9780471160199|Online ISBN: 9780471220572|DOI: 10.1002/0471220574
Tuvia S, Pelled D, Marom K, Salama P, Levin-Arama M, Karmeli I, Idelson G H, Landau I, Mamluk R. A novel suspension formulation enhances intestinal absorption of macromolecules via transient and reversible transport mechanisms. Pharm Res. 2014 August; 31(8):2010-21. doi: 10.1007/s11095-014-1303-9. Epub 2014 Feb. 21. PMID: 24558008; PMCID: PMC4153969.
US 2013-029907 A1
WO 2016/011208 A1
WO 2017/011820 A2
WO 2018/089693 A2
WO 2018/022937 A1
WO 2018/136646 A1
WO 2022/109328 A1
WO 2013/063468 A1
WO 2015/179438 A1

1. A peptide compound of formula (I): 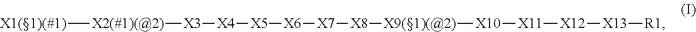
wherein
X1 is absent or is a Butyl moiety and
when X1 is a Butyl moiety, then (§ 1) represent an alkyl bond between X1 and X9 and (#1) represents an alkyl bond between X1 and X2, or
when X1 is absent, then (@2) represents an amide bond between X2 and X9,
X2 is an amino acid residue selected among a residue of Add, Ade, Ahd, Aoa, Atd, Atea, Aud, Dad, Dae, Dec, Dod, Dpa, Kme, Kme(αAc), Lys(αAc), Nmapt, Nmha and Oca;
X3 is an amino acid residue selected among a residue of Nak, Trp, Wdm, Wim, Wme, Wcl and Wfl;
X4 is an amino acid residue selected among a residue of Aib, Gln, Glu, Ile, Iva, Leu, Lys, Lys(Ac), Mle, Mly, Mva, Phe, Thr, Trp and Val;
X5 is an amino acid residue selected among a residue of Aib, Mkdm, Mle, and Thp;
X6 is an amino acid residue selected among a residue of Trp, Tyr, Yae, Yde and Yme;
X7 is an amino acid residue selected among a residue of Nak, Nal and Trp;
X8 is an amino acid residue selected among a residue of Aib, Cba, Cit, Gln, Leu, Lys, Lys(Ac), Mle, Mly, D-Mkdm, Mkdm, Thp, D-Trp and D-Tza;
X9 is an amino acid residue selected among a residue of Asp, Glu, Kme, Lys and Orn;
X10 is an amino acid residue selected among a residue of Ala, Asn, Gly and Ser;
X11 is an amino acid residue selected among a residue of Ala, Asn, Bal, Gly, Hol, Hph, His, Ile, Iva, Leu, Mhis, Pal, Pyal, PyEA, Val, and their corresponding D-forms;
X12 is absent or is an amino acid residue selected among a residue of Bal, Dnmy, Ile, Lys, Mep, Mhis and Sar;
X13 is absent or is a residue of Lys; and,
R1 is absent or is selected among —NH₂, —OH and —N(C₂H₅)₂,
or a salt or a solvate thereof.

2. The peptide compound according to claim 1, wherein
X1 is a Butyl moiety, and X9 is a residue of Kme, or
X1 is absent, X2 is an amino acid residue selected among a residue of Dad, Dae, Dec, Dod, Dpa and Oca, and X9 is an amino acid residue selected among a residue Kme, Lys, and Orn, or
X1 is absent, X2 is an amino acid residue selected among a residue of Dec, Dod, Dpa and Oca, and X9 is an amino acid residue selected among a residue of Kme and Lys, or
X1 is absent, X2 is an amino acid residue selected among a residue of Add, Ade, Ahd, Aoa, Atd, Atea, Aud, Kme, Kme(αAc), Lys(αAc), Nmapt and Nmha, and X9 is an amino acid residue selected among a residue of Asp and Glu, or
X1 is absent, X2 is an amino acid residue selected among a residue of Add, Ade, Ahd, Aoa, Atd, Atea and Aud, and X9 is a residue of Glu.

3. The peptide compound according to claim 1, wherein
X2 is an amino acid residue selected among a residue of Add, Ade, Ahd, Aoa, Atd, Atea, Aud, Dec, Dod, Dpa, Kme, Kme(αAc), Nmapt, Nmha and Oca;
X3 is an amino acid residue selected among a residue of Trp, Wdm, Wim, and Wme;
X4 is an amino acid residue selected among a residue of Gln, Leu and Lys(Ac);
X5 is an amino acid residue selected among a residue of Aib and Thp;
X6 is a residue of Yde;
X7 is a residue of Nal;
X8 is an amino acid residue selected among a residue of Aib, Mkdm and Thp;
X9 is an amino acid residue selected among a residue of Glu, Kme and Lys;
X10 is an amino acid residue selected among a residue of Asn, Gly and Ser;
X11 is an amino acid residue selected among a residue of Pal and PyEA;
X12 is absent or is a residue of Sar;
X13 is absent; and,
R1 is absent or is —NH₂.

4. The peptide compound according to claim 3, wherein
X10 represents Asn; or,
X10 represents an amino acid residue selected among a residue of Gly and Ser and X12 represents Sar; and
R1 represents NH₂, or
R1 represents NH₂ and
when X1 is a butyl moiety, X2 represents an amino acid residue selected among a residue of Kme, Nmapt and Nmha; or
when X1 is absent, X2 represents an amino acid residue selected among Add, Ade, Aoa, Atd, Atea, Aud, Dec, Dod and Oca.

5. The peptide compound according to claim 1, wherein
X2 is an amino acid residue selected among a residue of Add, Ade, Ahd, Aoa, Atd, Atea, Aud, Dec, Dod, Dpa, Kme, Kme(αAc), Nmapt, Nmha and Oca;
X3 is an amino acid residue selected among a residue of Trp, Wim and Wme;
X4 is an amino acid residue selected among a residue of Gln, Leu and Lys(Ac);
X5 is an amino acid residue selected among a residue of Aib and Thp;
X6 is a residue of Yde;
X7 is a residue of Nal;
X8 is an amino acid residue selected among a residue of Aib, Mkdm and Thp;
X9 is an amino acid residue selected among a residue of Glu, Kme and Lys;
X10 is an amino acid residue selected among a residue of Asn, Gly and Ser;
X11 is an amino acid residue selected among a residue of Pal and PyEA;
X12 is absent or is a residue of Sar;
X13 is absent;
R1 is absent or is —NH₂,
wherein
when X2 is a residue of Nmapt, X11 is a residue of PyEA, and X4 is a residue of Gln, then X10 is not a residue of Gly, or
when X2 is a residue of Nmapt, X11 is a residue of PyEA, and X10 is a residue of Gly, then X4 is not a residue of Gln, or
when X2 is a residue of Nmha and X11 is a residue of PyEA, then X4 is not a residue of Lys(Ac).

6. The peptide compound according to claim 1, wherein
X2 is an amino acid residue selected among a residue of Add, Ade, Ahd, Aoa, Atd, Atea, Aud, Dec, Dod, Dpa, Kme, Kme(αAc), Nmapt, Nmha and Oca;
X3 is an amino acid residue selected among a residue of Trp, Wim and Wme;
X4 is an amino acid residue selected among a residue of Gln, Leu and Lys(Ac);
X5 is an amino acid residue selected among a residue of Aib and Thp;
X6 is a residue of Yde;
X7 is a residue of Nal;
X8 is an amino acid residue selected among a residue of Aib, Mkdm and Thp;
X9 is an amino acid residue selected among a residue of Glu, Kme and Lys;
X10 is an amino acid residue selected among a residue of Asn, Gly and Ser;
X11 is an amino acid residue selected among a residue of Pal and PyEA;
X12 is absent or is a residue of Sar;
X13 is absent; and
R1 is absent or is —NH₂;
wherein
when X2 is a residue of Nmapt, X11 is a residue of PyEA, and X4 is a residue of Gln, then X10 is not a residue of Gly, or
when X2 is a residue of Nmapt, X11 is a residue of PyEA, and X4 is a residue of Lys(Ac), then X10 is not a residue of Ser, or
when X2 is a residue of Nmapt, X11 is a residue of PyEA, and X10 is a residue of Gly, then X4 is not a residue of Gln, or
when X2 is a residue of Nmha, then X11 is not a residue of PyEA.

7. The peptide compound according to claim 1, wherein
X2 is an amino acid residue selected among a residue of Add, Ade, Ahd, Aoa, Atd, Atea, Aud, Dec, Dod, Kme, Kme(αAc), Nmapt, Nmha and Oca;
X3 is an amino acid residue selected among a residue of Trp, Wim and Wme;
X4 is an amino acid residue selected among a residue of Gln, Leu and Lys(Ac);
X5 is an amino acid residue selected among a residue of Aib and Thp;
X6 is a residue of Yde;
X7 is a residue of Nal;
X8 is an amino acid residue selected among a residue of Aib, Mkdm and Thp;
X9 is an amino acid residue selected among a residue of Glu, Kme and Lys;
X10 is an amino acid residue selected among a residue of Asn, Gly and Ser;
X11 is an amino acid residue selected among a residue of Pal and PyEA;
X12 is absent or is a residue of Sar;
X13 is absent; and
R1 is absent or is —NH₂;
wherein
when X2 is a residue of Nmapt, then X11 is not PyEA, or
when X2 is a residue of Nmapt and X11 is a residue of PyEA, then X3 is a residue of Wim,
X4 is a residue of Gln and X10 is a residue of Ser, or
when X2 is a residue of Nmha, then X11 is not a residue of PyEA, or
when X2 is a residue of Aoa or Dec, then X4 is not a residue of Leu.

8. The peptide compound according to claim 1, wherein
X2 is an amino acid residue selected among a residue of Add, Ade, Aoa, Atd, Atea, Aud, Dec, Dod, Kme, Kme(αAc), Nmapt, Nmha and Oca;
X3 is an amino acid residue selected among a residue of Trp, Wim and Wme;
X4 is an amino acid residue selected among a residue of Gln, Leu and Lys(Ac);
X5 is an amino acid residue selected among a residue of Aib and Thp;
X6 is a residue of Yde;
X7 is a residue of Nal;
X8 is an amino acid residue selected among a residue of Aib, Mkdm and Thp;
X9 is an amino acid residue selected among a residue of Glu, Kme and Lys;
X10 is an amino acid residue selected among a residue of Asn, Gly and Ser;
X11 is a residue of Pal;
X12 is a residue of Sar;
X13 is absent; and
R1 is —NH₂;
wherein,
when X2 is a residue of Nmapt and X3 is a residue of Wme, then X8 is not a residue of a residue of Mkdm, or
when X2 is a residue of Aoa, then X4 is not a residue of a residue of Leu.

9. The peptide compound according to claim 1, wherein the peptide compound is represented by SEQ ID NO: 1 to 50, or a salt or a solvate thereof.

10. The peptide compound according to claim 9, wherein the peptide compound is represented by a sequence selected among:
SEQ ID NO: 1-22, 24, 26-48 and 50, or a salt or a solvate thereof; or
SEQ ID NO: 1-21, 24, 26-36, 38-48 and 50, or a salt or a solvate thereof; or
SEQ ID NO: 1-8, 10-14, 16, 18-21, 24, 26-36, 39-47 and 50, or a salt or a solvate thereof; or
SEQ ID NO: 1-2, 4-8, 10-14, 16, 18-21, 24, 26-35, 39-44, 46-47 and 50, or a salt or a solvate thereof; or
SEQ ID NO: 1-2, 4-5, 7-8, 10-14, 16, 18-21, 26, 28-29, 31-35, 39-40, 43-44, 46-47 and 50, or a salt or a solvate thereof; or
SEQ ID NO: 1-2, 4-5, 7-8, 10-14, 16, 18-21, 31-35, 39, 44, 46-47 and 50, or a salt or a solvate thereof; or
SEQ ID NO: 4, 11, 16, 18, 19 and 50, or a salt or a solvate thereof.

11. The peptide compound according to claim 1, wherein said peptide compound inhibits the IL-23-induced STAT3 phosphorylation with an IC₅₀ of about 50 nM or less, or of about 25 nM or less, or of about 15 nM or less, or of about 10 nM or less, or of about 5 nM or less, or of about 3 nM or less, or of about 2 nM or less, or of about 1 nM or less.

12. The peptide compound according to claim 1, wherein said peptide compound has a protease stability measured as a percentage of remaining of said peptide compound in simulated intestinal fluids at 60 min, 37° C., of at least 50%.

13. (canceled)

14. (canceled)

15. A pharmaceutical composition comprising at least one peptide compound according to claim 1, or a pharmaceutically acceptable salt or a solvate thereof, and at least one pharmaceutically acceptable excipient.

16. A method for treating and/or preventing an autoimmune or an inflammatory disease, the method comprising at least a step of administering to said subject a peptide compound according to claim 1.

17. (canceled)

18. The method according to claim 16, wherein the autoimmune or inflammatory disease is selected among inflammatory bowel disease, such as Crohn's disease and ulcerative colitis, psoriasis, psoriatic arthritis and hidradenitis suppurativa.


SEQUENCE LISTING (51 sequences)

SEQ ID NO: 1 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Aspartic acid in position 10.
XNTWQLXXXDNN

SEQ ID NO: 2 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Aspartic acid in position 10.
XNTWQAXXXDNN

SEQ ID NO: 3 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 2-naphthyl-L-alanine (Nal)
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Aspartic acid in position 10.
XNTWQRXXXDNN

SEQ ID NO: 4 - protein, 12 aa, synthetic construct
  SITE 1: note = X in position 1 is D-Lysine; note = Acetylation of N-terminus
  SITE 8: note = X in position 8 is 2-naphthyl-L-alanine.
  SITE 9: note = X in position 9 is 2-Aminoisobutyric acid.
  CROSSLNK 1..10: note = The amine moiety of the side chain of D- Lysine in position 1 forms a lactam bond with the carboxylic moiety of the side chain of Aspartic acid in position 10.
XNTWQRYXXDNN

SEQ ID NO: 5 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Glutamic acid in position 10.
XNTWQRXXXENN

SEQ ID NO: 6 - protein, 12 aa, synthetic construct
  SITE 1: note = X in position 1 is D-Aspartic acid; note = Acetylation of N-terminus
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of D-Aspartic acid in position 1 forms a lactam bond with the amine moiety of the side chain of Orn in position 10.
XNTWQRXXXXNN

SEQ ID NO: 7 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Aspartic acid in position 10.
XNTWQLXXXDNN

SEQ ID NO: 8 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D- Lysine in position 1 forms a lactam bond with the carboxylic moiety of the side chain of Aspartic acid in position 10.
XNTWQAXXXDNN

SEQ ID NO: 9 - protein, 12 aa, synthetic construct
  SITE 1: note = Lysine in position 1 has an acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of Lysine in position 1 forms a lactam bond with the carboxylic moiety of the side chain of Aspartic acid in position 10.
KNTWQRXXXDNN

SEQ ID NO: 10 - protein, 12 aa, synthetic construct
  SITE 1: note = Acetylation of N-terminus.; note = X in position 1 is D-Lysine
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 9: note = Lys in position 9 has an acetylation of N-terminus.
  CROSSLNK 1..10: note = The amine moiety of the side chain of D- Lysine in position 1 forms a lactam bond with the carboxylic moiety of the side chain of Aspartic acid in position 10.
XNTXQRXXKDNN

SEQ ID NO: 11 - protein, 12 aa, synthetic construct
  SITE 1: note = X in position 1 is D-Aspartic acid; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The carboxylic moiety of the side chain of D-Aspartic acid in position 1 forms a lactam bond with the amine moiety of the side chain of Lysine in position 10.
XNTWQRXXXKNN

SEQ ID NO: 12 - protein, 12 aa, synthetic construct
  SITE 1: note = X in position 1 is D-Aspartic acid; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The carboxylic moiety of the side chain of D-Aspartic acid in position 1 forms a lactam bond with the amine moiety of the side chain of Lysine in position 10.
XNTWQRXXXKNN

SEQ ID NO: 13 - protein, 12 aa, synthetic construct
  SITE 1: note = X in position 1 is D-Aspartic acid; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of D-Aspartic acid in position 1 forms a lactam bond with the amine moiety of the side chain of Orn in position 10.
XNTWQRXXXXNN

SEQ ID NO: 14 - protein, 12 aa, synthetic construct
  SITE 1: note = X in position 1 is D-Lysine; note = Acetylation of N-terminus.
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Aspartic acid in position 10.
XNTWQRYXXDNN

SEQ ID NO: 15 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Aspartic acid in position 10.
XNTWQRXXXDNN

SEQ ID NO: 16 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Glutamic acid in position 10.
XNTWQRXXXENN

SEQ ID NO: 17 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = Lysine in position 9 has an acetylation of N-terminus.
  CROSSLNK 1..10: note = The amine moiety of the side chain of D-Lysine in position 1 forms a lactam bond with the carboxylic moiety of the side chain of Asp in position 10.
XNTWQRXXKDNN

SEQ ID NO: 18 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is L-citrulline (Cit).
  CROSSLNK 1..10: note = The amine moiety of the side chain of D-Lysine in position 1 forms a lactam bond with the carboxylic moiety of the side chain of Asp in position 10.
XNTWQRXXXDNN

SEQ ID NO: 19 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  CROSSLNK 1..10: note = The amine moiety of the side chain of D-Lysine in position 1 forms a lactam bond with the carboxylic moiety of the side chain of Asp in position 10.
XNTWQRXXQDNN

SEQ ID NO: 20 - protein, 12 aa, synthetic construct
  SITE 1: note = X in position 1 is D-Lysine; note = Acetylation of N-terminus
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Asp in position 10.
XNTWQVXXXDNN

SEQ ID NO: 21 - protein, 12 aa, synthetic construct
  SITE 1: note = X in position 1 is D-Lysine; note = Acetylation of N-terminus.
  SITE 6: note = X in position 6 is 2-Aminoisobutyric acid (Aib).
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Asp in position 10.
XNTWQXXXXDNN

SEQ ID NO: 22 - protein, 12 aa, synthetic construct
  SITE 1: note = X in position 1 is D-Ornithine; note = Acetylation of N-terminus.
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is 2-Aminoisobutyric acid (Aib).
  CROSSLNK 1..10: note = The amine moiety of the side chain of D-Ornithine in position 1 forms a lactam bond with the carboxylic moiety of the side chain of Glutamic acid in position 10.
XNTWQRXXXENN

SEQ ID NO: 23 - protein, 12 aa, synthetic construct
  SITE 1: note = X in position 1 is D-Lysine; note = Acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 1-naphthyl-L-alanine (Nak).
  SITE 9: note = X in position 9 is alpha-methyl-L-leucine (Mle).
  CROSSLNK 1..10: note = The amine moiety of the side chain of D-Lysine in position 1 forms a lactam bond with the carboxylic moiety of the side chain of Asp in position 10.
XNTXQRXXXDNN

SEQ ID NO: 24 - protein, 12 aa, synthetic construct
  SITE 1: note = X in position 1 is D-Lysine; note = Acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is alpha-methyl-L-leucine (Mle).
  CROSSLNK 1..10: note = The amine moiety of the side chain of D-Lysine in position 1 forms a lactam bond with the carboxylic moiety of the side chain of Asp in position 10.
XNTXQRXXXDNN

SEQ ID NO: 25 - protein, 12 aa, synthetic construct
  SITE 1: note = X in position 1 is D-Lysine; note = Acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is D-beta-(4-thiazolyl)-alanine (DTza).
  CROSSLNK 1..10: note = The amine moiety of the side chain of D-Lysine in position 1 forms a lactam bond with the carboxylic moiety of the side chain of Asp in position 10.
XNTXQRXXXDNN

SEQ ID NO: 26 - protein, 11 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Asp in position 10.
XNTWQRXXXDN

SEQ ID NO: 27 - protein, 12 aa, synthetic construct
  SITE 1: note = X in position 1 is D-Ornithine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D-Ornithine in position 1 forms a lactam bond with the carboxylic moiety of the side chain of Asp in position 10.
XNTWQRXXXDNN

SEQ ID NO: 28 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = Lysine in position 9 has an acetylation of N-terminus.
  CROSSLNK 1..10: note = The amine moiety of the side chain of D-Lysine in position 1 forms a lactam bond with the carboxylic moiety of the side chain of Asp in position 10.
XNTWQRXXKDNN

SEQ ID NO: 29 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is L-citrulline (Cit).
  CROSSLNK 1..10: note = The amine moiety of the side chain of D-Lysine in position 1 forms a lactam bond with the carboxylic moiety of the side chain of Asp in position 10.
XNTWQRXXXDNN

SEQ ID NO: 30 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  CROSSLNK 1..10: note = The amine moiety of the side chain of D-Lysine in position 1 forms a lactam bond with the carboxylic moiety of the side chain of Asp in position 10.
XNTWQRXXQDNN

SEQ ID NO: 31 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Asp in position 10.
XNTWQVXXXDNN

SEQ ID NO: 32 - protein, 12 aa, synthetic construct
  SITE 1: note = X in position 1 is D-Lysine; note = Acetylation of N-terminus.
  SITE 6: note = X in position 6 is 2-Aminoisobutyric acid (Aib).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of the side chain of D-Lysine in position 1 forms a lactam bond with the carboxylic moiety of the side chain of Asp in position 10.
XNTWQXXXXDNN

SEQ ID NO: 33 - protein, 12 aa, synthetic construct
  SITE 1: note = X in position 1 is Aspartic acid; note = Acetylation of N-terminus.
  SITE 4: note = X in position 4 is 1-methyl-L-tryptophan (Wme).
  SITE 8: note = X in position 8 is 2-naphthyl-L-alanine (Nal).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of Aspartic acid in position 1 forms a lactam bond with the amine moiety of the side chain of Ornithine in position 10.
XNTXQRYXXXNN

SEQ ID NO: 34 - protein, 12 aa, synthetic construct
  SITE 1: note = Acetylation of N-terminus.; note = X in position 1 is Aspartic acid
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of Aspartic acid in position 1 forms a lactam bond with the amine moiety of the side chain of Ornithine in position 10.
XNTXQRXXXXNN

SEQ ID NO: 35 - protein, 12 aa, unidentified
  SITE 1: note = Glutamic acid in position 1 has an acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of Glutamic acid in position 1 forms a lactam bond with the amine moiety of the side chain of Ornithine in position 10.
ENTXQRXXXXNN

SEQ ID NO: 36 - protein, 12 aa, synthetic construct
  SITE 1: note = X in position 1 is Aspartic acid; note = Acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 9: note = X in position 9 is 2-Aminoisobutyric acid (Aib).
  SITE 10: note = X in position 10 is Ornithine (Orn).
  CROSSLNK 1..10: note = The carboxylic moiety of the side chain of Aspartic acid in position 1 forms a lactam bond with the amine moiety of the side chain of Ornithine in position 10.
XNTXQRXWXXNN

SEQ ID NO: 37 - protein, 12 aa, synthetic construct
  SITE 1: note = X in position 1 is Aspartic acid; note = Acetylation of N-terminus.
  SITE 4: note = X in position 4 is 1-methyl-L-tryptophan (Wme).
  SITE 7: note = X in position 7 is 4-methoxy-L-phenylalanine (Yme).
  SITE 8: note = X in position 8 is 2-naphthyl-L-alanine (Nal).
  SITE 10: note = X in position 10 is Ornithine (Orn).
  CROSSLNK 1..10: note = The carboxylic moiety of the side chain of Aspartic acid in position 1 forms a lactam bond with the amine moiety of the side chain of Ornithine in position 10.
XNTXQRXXLXNN

SEQ ID NO: 38 - protein, 12 aa, synthetic construct
  SITE 1: note = X in position 1 is Ornithine; note = Acetylation of N-terminus.
  SITE 7: note = X in position 7 is 4-aminoethoxy-L-phenylalanine (Yae).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The carboxylic moiety of the side chain of Glutamic acid in position 10 forms a lactam bond with the amine moiety of the side chain of Ornithine in position 1.
XNTWQRXXXENN

SEQ ID NO: 39 - protein, 12 aa, synthetic construct
  SITE 1: note = X in position 1 is D-Lysine; note = Acetylation of N-terminus.
  SITE 6: note = X in position 6 is (2S)-2-amino-5-guanidino-butyric acid (Agb).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The carboxylic moiety of the side chain of Aspartic acid in position 10 forms a lactam bond with the amine moiety of the side chain of D-Lysine in position 1.
XNTWQXXXXDNN

SEQ ID NO: 40 - protein, 12 aa, synthetic construct
  SITE 1: note = X in position 1 is 6-aminohexanoic acid (Ahx).
  SITE 6: note = X in position 6 is (2S)-2-amino-5-guanidino-butyric acid (Agb).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amino group of Ahx in position 1 forms a lactam bond with the carboxylic moiety of the side chain of Glutamic acid in position 10.
XNTWQXXXXENN

SEQ ID NO: 41 - protein, 12 aa, synthetic construct
  SITE 1: note = X in position 1 is Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 4-aminotetrahydropyran-4-carboxylic acid (Thp).
  CROSSLNK 1..10: note = The carboxylic moiety of the side chain of Aspartic acid in position 10 forms a lactam bond with the amine moiety of the side chain of Lysine in position 1.
XNTWQRXXXDNN

SEQ ID NO: 42 - protein, 12 aa, synthetic construct
  SITE 1: note = X in position 1 is Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 4-aminotetrahydropyran-4-carboxylic acid (Thp).
  CROSSLNK 1..10: note = The carboxylic moiety of the side chain of Aspartic acid in position 10 forms a lactam bond with the amine moiety of the side chain of Lysine in position 1.
XNHWQRXXXDNN

SEQ ID NO: 43 - protein, 12 aa, synthetic construct
  SITE 1: note = X in position 1 is 6-aminohexanoic acid (Ahx).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amino group of Ahx in position 1 forms a lactam bond with the carboxylic moiety of the side chain of Aspartic acid in position 10.
XNTWQAXXXDNN

SEQ ID NO: 44 - protein, 12 aa, synthetic construct
  SITE 1: note = X in position 1 is 5-aminohpentanoic acid (Apt).
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amino group of Apt in position 1 forms a lactam bond with the carboxylic moiety of the side chain of Aspartic acid in position 10.
XNTWQAXXXDNN

SEQ ID NO: 45 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of D-Lysine in position 1 forms a lactam bond with the carboxylic moiety of the side chain of Aspartic acid in position 10.
XRTWQAXXXDNN

SEQ ID NO: 46 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of D-Lysine in position 1 forms a lactam bond with the carboxylic moiety of the side chain of Aspartic acid in position 10.
XNRWQAXXXDNN

SEQ ID NO: 47 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  CROSSLNK 1..10: note = The amine moiety of D-Lysine in position 1 forms a lactam bond with the carboxylic moiety of the side chain of Aspartic acid in position 10.
XNTWQAXXRDNN

SEQ ID NO: 48 - protein, 12 aa, unidentified
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of D-Lysine in position 1 forms a lactam bond with the carboxylic moiety of the side chain of Aspartic acid in position 10.
XNTWQAXXXDNA

SEQ ID NO: 49 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of D-Lysine in position 1 forms a lactam bond with the carboxylic moiety of the side chain of Aspartic acid in position 10.
XNTWQAXXXDAN

SEQ ID NO: 50 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 2-Aminoisobutyric acid (Aib).
  CROSSLNK 1..10: note = The amine moiety of D-Lysine in position 1 forms a lactam bond with the carboxylic moiety of the side chain of Aspartic acid in position 10.
XNTWQAXXXDAA

SEQ ID NO: 51 - protein, 12 aa, synthetic construct
  SITE 1: note = X in position 1 is D-Lysine; note = Acetylation of N-terminus.
  SITE 7: note = X in position 7 is 4-methoxy-L-phenylalanine (Yme).
  SITE 8: note = X in position 8 is 2-naphthyl-L-alanine (Nal).
  SITE 9: note = X in position 9 is 4-aminotetrahydropyran-4-carboxylic acid (Thp).
  CROSSLNK 1..10: note = The amine moiety of D-Lysine in position 1 forms a lactam bond with the carboxylic moiety of the side chain of Aspartic acid in position 10.
XNTWQAXXXDNN